(19) United States
(12) Patent Application Publication
(10) Pub. No.: US 2026/0224796 A1
(43) Pub. Date: Aug. 6, 2026

(54) COMPACT NEGATIVE PRESSURE WOUND THERAPY SYSTEM

(71) Applicant: Xairio, Inc., Chagrin Falls, OH (US)

(72) Inventors: John Buan, Maple Grove, MN (US); Edward Armstrong, Chagrin Falls, OH (US)

(21) Appl. No.: 19/463,764

(22) Filed: Jan. 29, 2026

Related U.S. Application Data
(60) Provisional application No. 63/753,098, filed on Feb. 3, 2025.

(51) Int. Cl. A61M 1/00; A61M 39/22

(52) U.S. Cl. CPC A61M 1/912; A61M 1/915; A61M 1/918; A61M 1/962; A61M 1/985; A61M 39/22; A61M 2202/0014; A61M 2202/04; A61M 2205/3344; A61M 2207/00; A61M 2209/088; A61M 2210/04

(57) ABSTRACT

Aspects include a negative pressure wound therapy system and methods for manufacturing a negative pressure wound therapy system. The system comprises a dressing configured to be positioned on a wound site, a hub configured to have aspirational attachment to the dressing, and a pressure engine pre-evacuated to a sub-atmospheric pressure and configured to have aspirational attachment the hub such that the pressure engine is configured to aspirate fluid away from the dressing via a fluid channel extending from the dressing, through the hub, to the pressure engine. Sub-atmospheric pressure in the pressure engine can be achieved by evacuating air from and then sealing the pressure engine, forming the pressure engine in a sub-atmospheric pressure environment and then sealing the pressure engine, or forming the pressure engine in a first environment at sub-atmospheric pressure relative to a second environment and moving the pressure engine to the second environment for use.

### CROSS-REFERENCE TO RELATED APPLICATIONS

This application claims the benefit under 35 U.S.C. §119(e) of U.S. Provisional Patent Application No. 63/753,098 filed Feb. 3, 2025, the content of which is incorporated by reference herein in its entirety.

### TECHNICAL FIELD

The description relates generally to negative pressure wound therapy systems, including, for example, methods for using and manufacturing for negative pressure wound therapy systems.

### BACKGROUND

Negative pressure wound therapy (NPWT) is a method of treating wounds that involves applying negative (or, alternatively, sub-atmospheric) pressure to a wound site to promote healing. NPWT can be used for various types of wounds, such as chronic ulcers, surgical incisions, traumatic injuries, and burns.

### BRIEF SUMMARY

In an aspect, a negative pressure wound therapy system is provided. The negative pressure wound therapy system comprises a dressing. The dressing is configured to be positioned on a wound site. The dressing comprises a gasket. The gasket is configured to form an airtight seal surrounding the wound site. The dressing comprises a first adhesive. The first adhesive is configured to attach the dressing to skin surrounding the wound site. The negative pressure wound therapy system comprises a hub. The hub is configured to be aspirationally attached to the dressing. The negative pressure wound therapy system comprises a sealed pressure engine. The pressure engine is pre-evacuated to a sub-atmospheric pressure, relative to the absolute external atmospheric pressure in the environment of intended use. The pressure engine is configured to be aspirationally attached to the hub (e.g., with an airtight seal formed between the hub (and dressing) and the pressure engine) such that the pressure engine is configured to aspirate fluid away from the dressing via a fluid channel extending from the dressing, through the hub, to the pressure engine (i.e., the total absolute gas pressure of the system, if measured, would be considered to be at a therapeutic level, that is sub-atmospheric relative to the atmospheric pressure at the geographic location of use). The result is sub-atmospheric pressure (e.g., vacuum) is created and maintained over the wound site.

In aspects, at least a portion of an interior surface of the pressure engine may comprise (e.g., be coated with) a metallic film. In aspects, at least a portion of an exterior surface of the pressure engine may comprise (e.g., be coated with) a metallic film.

In aspects, the dressing may comprise a drape. The drape may comprise a first side configured to face toward the wound site and a second side configured to face away from the wound site. In aspects, at least a portion of the first side may comprise a metallic film. In aspects, the drape may comprise a portion extending beyond the gasket and including the first adhesive.

In aspects, the gasket may comprise a central opening. The dressing may comprise a wound contact structure disposed within the central opening of the gasket. The wound contact structure may define a sealed volume of the dressing. The wound contact structure may include elements that absorb wound exudate and other elements that serve as a wound contact layer. In aspects, a portion of the wound dressing within the central opening of the gasket may comprise an additional volume control element configured to mitigate shrinkage of the wound contact structure due to a pressure gradient existing between the external environment and the sealed volume of the dressing. In aspects, the dressing may include one or more absorptive pads, which may be designed to serve as the volume control element, but also absorb wound exudate.

In aspects, the negative pressure wound therapy system may comprise a tubing. The tubing may extend between the dressing and the hub. The tubing may be removably attached to both the dressing and the hub. The tubing may thereby aspirationally attach the hub to the dressing. In aspects, the dressing may comprise a tubing interface, and the tubing may be removably attached to the dressing via the tubing interface. In aspects, the hub may comprise a tubing interface, and tubing may be removably attached to the hub via the tubing interface. In aspects, the dressing may comprise a plurality of dressings. The tubing may comprise a plurality of tubings. Each tubing of the plurality of tubings may correspond to a respective one of the plurality of dressings. The hub may comprise a plurality of tubing interfaces. Each tubing interface of the plurality of tubing interfaces may be configured to be removably attached to a respective one of the plurality of tubings. Each tubing of the plurality of tubings may thereby aspirationally attach a respective one of the plurality of dressings to the hub. In aspects, the tubing may define a fluid channel extending an entire length of the tubing.

In aspects, the hub may comprise a second adhesive. The second adhesive may be configured to attach the hub to the skin at a different location than the wound site.

In aspects, the hub may be directly attached or integrated to the dressing.

In aspects, the pressure engine may be configured to be physically attached to the hub. At least one of the hub and the pressure engine may be configured to be selectively moved relative to the other of the hub and the pressure engine between (a) a first position in which the pressure engine is physically attached to the hub but not aspirationally attached to the hub and (b) a second position in which the pressure engine is both physically attached to the hub and aspirationally attached to the hub.

In aspects, the hub may comprise a pressure engine interface. The pressure engine may be removably attached to the hub via the pressure engine interface. The pressure engine may be configured to automatically aspirate fluid away from the dressing in response to being aspirationally attached to the pressure engine interface of the hub. In aspects, the pressure engine may comprise a hub interface. The hub interface may be configured to at least partially form the fluid channel with the pressure engine interface. In aspects, one of the pressure engine interface of the hub and the hub interface of the pressure engine may include a seal. The other of the pressure engine interface of the hub and the hub interface of the pressure engine may include an engagement feature. The pressure engine interface of the hub (e.g., seal or engagement feature) and the hub interface of the pressure engine (e.g., seal or engagement feature) may cooperate to form an airtight seal between the pressure engine and the hub, such that a volume of the pressure engine and a volume of the hub can be regarded as a single enclosed volume. The engagement feature may be configured to puncture the seal such that the pressure engine is configured to automatically aspirate fluid away from the dressing in response to puncturing the seal. In aspects, one of the pressure engine interface of the hub and the hub interface of the pressure engine may include a valve or a membrane. The other of the pressure engine interface of the hub and the hub interface of the pressure engine may include an engagement feature. The engagement feature may be configured to open the valve or puncture the membrane, such that the pressure engine is configured to automatically aspirate fluid away from the dressing in response to opening the valve or puncturing the membrane. In equilibrium, a short time after opening the valve or puncturing the membrane, the combined system of the pressure engine and the hub (e.g., and the dressing), may now be configured to function as a single, sealed volume, and may have a total absolute pressure which is sub-atmospheric relative to the atmospheric pressure at the geographic location of use.

In aspects, the pressure engine may automatically aspirate fluid away from the dressing in response to attachment to the hub.

In aspects, the hub may comprise one or more guides disposed atop the hub for aligning the pressure engine and the hub. In aspects, the pressure engine may comprise one or more guides disposed beneath the pressure engine for aligning the pressure engine and the hub.

In aspects, the negative pressure wound therapy system may be wearable by a patient.

In another aspect, a method for applying the negative pressure wound therapy system is provided. The method comprises positioning the dressing on the wound site such that the gasket forms an airtight seal surrounding the wound site and the first adhesive attaches the dressing to skin surrounding the wound site. The method comprises aspirationally attaching the hub to the dressing such that an airtight seal is formed between the hub and the dressing. The method comprises aspirationally attaching the pressure engine to the hub such that an airtight seal is formed between the pressure engine and the hub. The method comprises aspirating fluid away from the dressing via the fluid channel extending from the dressing, through the hub, to the pressure engine.

In aspects, the pressure engine may be a first pressure engine. The method may comprise aspirationally detaching the first pressure engine from the hub after aspirating fluid away from the dressing using the first pressure engine. The method may comprise aspirationally attaching a second pressure engine pre-evacuated to a sub-atmospheric pressure to the hub. The method may comprise aspirating, using the second pressure engine, fluid away from the dressing.

In aspects, the hub may comprise a second adhesive. In aspects, the method may comprise attaching the hub to the skin at a different location than the wound site via the second adhesive.

In aspects, the method may comprise directly attaching the hub to the dressing.

In aspects, the method may comprise selectively moving at least one of the hub and the pressure engine relative to the other of the hub and the pressure engine between (a) a first position in which the pressure engine is physically attached to the hub but not aspirationally attached to the hub and (b) a second position in which the pressure engine is both physically attached to the hub and aspirationally attached to the hub.

In another aspect, a method for manufacturing a negative pressure wound therapy system is provided. The method comprises forming a pressure engine having an internal chamber in an environment. The method comprises evacuating air from the internal chamber of the pressure engine such that the internal chamber of the pressure engine is at a sub-atmospheric pressure relative to the external atmospheric pressure in the environment. The method comprises sealing the internal chamber of the pressure engine so as to maintain the internal chamber of the pressure engine at the sub-atmospheric pressure.

In another aspect, another method for manufacturing a negative pressure wound therapy system is provided. The method comprises creating or providing a controlled pressure environment. The method comprises sealing a pressure engine having an internal chamber in the controlled pressure environment such that the pressure of the internal chamber of the sealed pressure engine is at a sub-atmospheric pressure equivalent to the air pressure of the controlled pressure environment.

In another aspect, another method for manufacturing a negative pressure wound therapy system is provided. The method comprises sealing a pressure engine having an internal chamber in a first environment such that the internal chamber of the pressure engine is at an internal pressure equivalent to the external atmospheric pressure in the first environment. The method further comprises moving the pressure engine from the first environment to a second environment different from the first environment, such that the internal pressure of the internal chamber of the pressure engine is sub-atmospheric relative to the external atmospheric pressure in the second environment.

In another aspect, another method for manufacturing a negative pressure wound therapy system is provided. The method provides an additionally or alternatively enables controlling the internal pressure of the pressure engine. The method comprises creating or providing a controlled pressure environment. The method comprises controlling a temperature in the controlled pressure environment. Controlling the temperature in the controlled pressure environment can, in aspects, include elevating the temperature relative to ambient temperature, thereby increasing the pressure in the controlled pressure environment. The method comprises sealing a pressure engine having an internal chamber in the controlled pressure environment. The method comprises raising the temperature in the controlled pressure environment above the ambient temperature of the atmosphere outside (e.g., external to) the controlled pressure environment. The method comprises, after sealing the pressure engine, removing the pressure engine from the controlled pressure environment, and allowing the pressure engine to subsequently cool to the ambient temperature, thereby causing the internal pressure of the pressure engine to reduce further upon cooling of the pressure engine.

Additional embodiments and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

While aspects described with respect to certain drawings and/or methods herein may differ, it will be understood by those skilled in the art, on review of the disclosures herein, that all aspects expressed with respect to methodologies may be implemented using systems disclosed herein, and all aspects described as to systems herein can be performed by methodologies disclosed herein, without departing from the scope or spirit of the innovation. Moreover, steps of methodologies and/or components of systems can be added, removed, consolidated, divided, re-ordered, or the like, without departing from the scope or spirit of the innovation. Embodiments, including any features and/or components thereof, may also be combined, in whole or in part, without departing from the scope or spirit of the innovation.

### BRIEF DESCRIPTION OF THE DRAWINGS

Certain features of the subject technology are set forth in the appended claims. However, for the purpose of explanation, several embodiments of the subject technology are set forth in the following figures.

FIG. 1 illustrates a perspective view of an exemplary wound therapy system in accordance with one or more non-limiting embodiments.

FIG. 2 illustrates an application of an exemplary wound therapy system in accordance with one or more non-limiting embodiments.

FIG. 3A illustrates an exploded view of an exemplary pressure engine and hub in accordance with one or more non-limiting embodiments.

FIG. 3B illustrates a top view of the exemplary pressure engine and hub of FIG. 3A in accordance with one or more non-limiting embodiments.

FIG. 3C illustrates a bottom view of the exemplary pressure engine and hub of FIG. 3A in accordance with one or more non-limiting embodiments.

FIG. 3D illustrates a perspective view of the exemplary pressure engine and hub of FIG. 3A in accordance with one or more non-limiting embodiments.

FIG. 3E illustrates a side view of the exemplary pressure engine and hub of FIG. 3A in accordance with one or more non-limiting embodiments.

FIG. 3F illustrates a side sectional view of the exemplary pressure engine and hub of FIG. 3A in accordance with one or more non-limiting embodiments.

FIG. 4 illustrates a perspective view of an exemplary dressing in accordance with one or more non-limiting embodiments.

FIG. 5A illustrates an exploded view of an exemplary pressure engine and hub in accordance with one or more non-limiting embodiments.

FIG. 5B illustrates a top view of the exemplary pressure engine and hub of FIG. 5A in accordance with one or more non-limiting embodiments.

FIG. 5C illustrates a bottom view of the exemplary pressure engine and hub of FIG. 5A in accordance with one or more non-limiting embodiments.

FIG. 5D illustrates a perspective view of the exemplary pressure engine and hub of FIG. 5A in accordance with one or more non-limiting embodiments.

FIG. 5E illustrates a side view of the exemplary pressure engine and hub of FIG. 5A in accordance with one or more non-limiting embodiments.

FIG. 5F illustrates a side sectional view of the exemplary pressure engine and hub of FIG. 5A in accordance with one or more non-limiting embodiments.

FIG. 6A illustrates a side view of an exemplary dressing in accordance with one or more non-limiting embodiments.

FIG. 6B illustrates a perspective view of the exemplary dressing of FIG. 6A in accordance with one or more non-limiting embodiments.

FIG. 7A graphically illustrates starting pressure required (in mmHg, along the vertical axis) vs. pressure engine volume (in mL, along the horizontal axis) for a 14” x 3.5” dressing and a pressure reduction of 100mmHg.

FIG. 7B graphically illustrates starting pressure required (in mmHg, along the vertical axis) vs. pressure engine volume (in mL, along the horizontal axis) for a 14” x 3.5” dressing and a target final pressure of 100mmHg.

FIG. 7C graphically illustrates starting pressure required (in mmHg, along the vertical axis) vs. pressure engine volume (in mL, along the horizontal axis) for a 14” x 3.5” dressing and a target final pressure of 100mmHg.

FIG. 7D graphically illustrates starting pressure required (in mmHg, along the vertical axis) vs. pressure engine volume (in mL, along the horizontal axis) for a 14” x 3.5” dressing and a target final pressure of 125mmHg.

FIG. 7E graphically illustrates starting pressure required (in mmHg, along the vertical axis) vs. pressure engine volume (in mL, along the horizontal axis) for a 14” x 3.5” dressing and a target final pressure of 125mmHg.

FIG. 7F graphically illustrates starting pressure required (in mmHg, along the vertical axis) vs. pressure engine volume (in mL, along the horizontal axis) for a 14” x 3.5” dressing and a target final pressure of 125mmHg.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other embodiments. In one or more embodiments, structures and components are shown in block diagram form in order to avoid obscuring the concepts of the subject technology.

NPWT may involve placing a dressing on the wound to seal the wound and attaching a suction tube to a vacuum source. The vacuum source can be adjusted to deliver a desired level of negative pressure to the wound, such as -40mmHg to -200mmHg depending on the wound characteristics and clinical goals. The negative pressure creates a sub-atmospheric pressure environment around the wound site, which may have beneficial effects on the wound healing process including, but not limited to, fluid removal, angiogenesis, lymph angiogenesis, and reduction of bacterial load in the wound tissue. For example, if atmospheric pressure is 760mmHg and the vacuum reduces pressure within the dressing to 660mmHg, the result may be a negative pressure of -100mmHg applied to the wound.

A dressing is a material or an assembly of materials that covers and/or protects a wound from potential infection, bleeding, or further damage. Dressings may vary in size, shape, thickness, and composition depending on the type and stage of the wound. Some types of wound dressings include gauze, bandages, hydrocolloids, alginates, foams, films, and meshes. Dressings may also have additional functions such as absorbing exudate, maintaining moisture, debriding necrotic tissue, or delivering drugs or other substances to the wound. A dressing may be configured for NPWT by including one or more components that create an airtight seal around the wound site, allowing for the application of negative pressure around the wound site with minimal leakage.

Non-limiting embodiments of the subject technology described herein provide for a NPWT system (also hereafter referred to as a wound therapy system or a therapy system) that may be worn by a patient. The NPWT system may include a wound dressing to be positioned on (e.g., to cover) the wound site on the patient’s skin and create an airtight seal around the wound site, so as to maintain the negative pressure provided by the pressure source. In some aspects, the NPWT system may include tubing to establish a fluid channel between the dressing, a hub, and/or a pressure engine, although other aspects are not so limited and the dressing, the hub, and/or the pressure engine may be attached to one another without the use of any intermediary tubing, as described herein.

An internal chamber of the pressure engine may be pre-evacuated to a sub-atmospheric pressure, relative to the intended environment of use, allowing the pressure engine to aspirate fluid away from the dressing and thus reduce the pressure within the dressing, when attached to the system and activated. For example, in some embodiments, a vacuum may be applied to the internal chamber of the pressure engine during the manufacturing process to pre-evacuate the pressure engine (e.g., to a sub-atmospheric pressure). In the same or other embodiments, a vacuum may be applied to the pressure engine during the fluid aspiration process to continuously evacuate the pressure engine (e.g., to a sub-atmospheric pressure). In other embodiments, the pressure engine may be formed in a sub-atmospheric environment or moved to result in the internal pressure of the pressure engine being sub-atmospheric relative to the external atmospheric pressure, as described herein. In other embodiments, the pressure engine may be sealed in a controlled pressure environment at elevated temperature and subsequently removed from the controlled pressure environment and allowed to cool to a lower, non-elevated (ambient) temperature to result in the internal pressure of the pressure engine to reduce to a sub-atmospheric pressure. In other embodiments, the nature of the gas(es) in the pressure engine can be controlled and the pressure engine can be sealed to provide a resulting pressure engine that maintains an internal sub-atmospheric pressure of a custom gas mixture. In aspects, the pressure engine may be at (e.g., evacuated to) any sub-atmospheric pressure relative to normal atmospheric pressure as desired to suit a particular application. For example, in some variations, the pressure engine may be at 95% of normal atmospheric pressure. In some variations, the pressure engine may be at 90% of normal atmospheric pressure. In some variations, the pressure engine may be at 85% of normal atmospheric pressure. In some variations, the pressure engine may be at 80% of normal atmospheric pressure. In some variations, the pressure engine may be at 75% of normal atmospheric pressure. In some variations, the pressure engine may be at 70% of normal atmospheric pressure. In some variations, the pressure engine may be at 80% of normal atmospheric pressure. In some variations, the pressure engine may be at 65% of normal atmospheric pressure. In some variations, the pressure engine may be at 55% of normal atmospheric pressure. In some variations, the pressure engine may be at 50% of normal atmospheric pressure. In some variations, the pressure engine may be at 45% of normal atmospheric pressure. In some variations, the pressure engine may be at 40% of normal atmospheric pressure. In some variations, the pressure engine may be at 35% of normal atmospheric pressure. In some variations, the pressure engine may be at 30% of normal atmospheric pressure. In some variations, the pressure engine may be at 25% of normal atmospheric pressure. In some variations, the pressure engine may be at 20% of normal atmospheric pressure. In some variations, the pressure engine may be at 15% of normal atmospheric pressure. In some variations, the pressure engine may be at 10% of normal atmospheric pressure. In some variations, the pressure engine may be at 5% of normal atmospheric pressure. In aspects, the pressure engine may be sealed or otherwise configured to maintain the internal chamber thereof at sub-atmospheric pressure for a predetermined length of time as desired to suit a particular application. For example, in some variations, the pressure engine may be sealed such that the sub-atmospheric pressure within the pressure engine is maintained for up to two years. In some variations, at least a portion of an interior surface and/or at least a portion of an exterior surface of the pressure engine may comprise a metallic film. In some variations, the pressure engine may be coated internally and/or externally with a metallic film to reduce diffusion of gases into the pressure engine that would increase the absolute pressure within the internal chamber of the pressure engine, thereby reducing the level of negative pressure, maintained by the device, relative to the atmospheric pressure at the geographic site of use. Coating or otherwise providing a metallic film on at least a portion of an interior surface and/or on at least a portion of an exterior surface of the pressure engine may increase the useful life (e.g., shelf life) of the pressure engine, including for up to two years.

In some embodiments, the pressure engine may be configured to adhere to the skin (e.g., via an adhesive), proximate to the dressing, thereby allowing the pressure engine to attach to the dressing with minimal tubing. In some embodiments, the pressure engine includes or attaches to a hub that adheres to the skin or the dressing, allowing for attachment to multiple dressings. In some embodiments, the hub may be configured to be directly attached to the dressing, such that when the pressure engine is physically attached to the hub, the hub and the pressure engine are attached to the dressing without the need for any tubing therebetween. In some embodiments, the pressure engine may be a simple, wearable, single-use system that may operate without the use of an active power source, such as a battery, or complex mechanical components, such as a motor. In this way, in some embodiments, the pressure engine may be configured to automatically aspirate fluid away from the dressing in response to attachment to the hub.

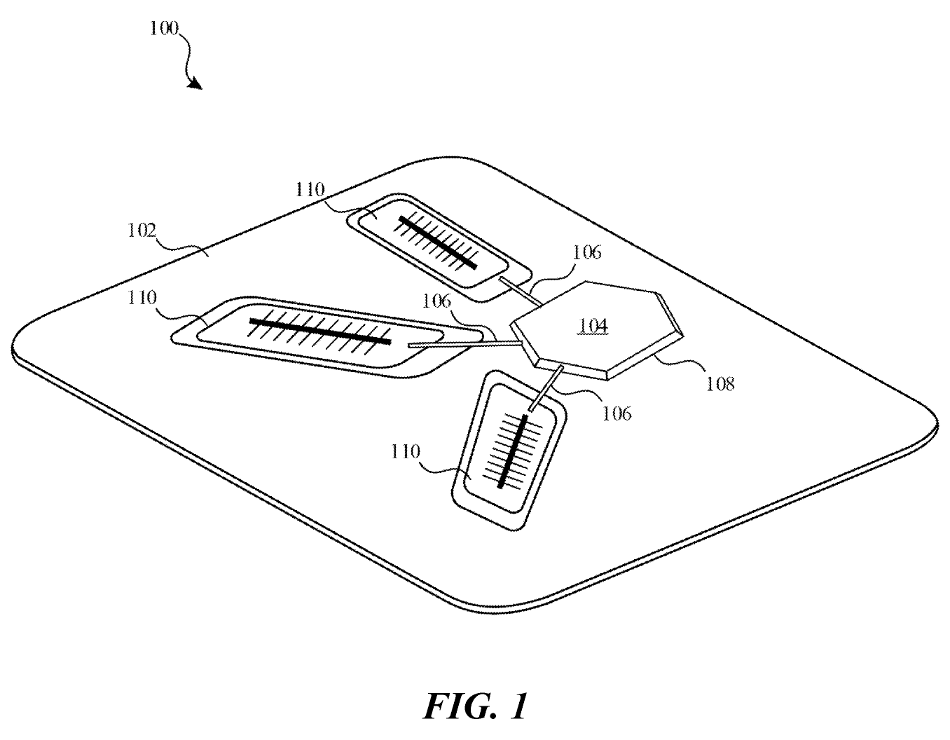

These and other embodiments are discussed below with reference to FIGS. 1-6. However, those skilled in the art will readily appreciate that the detailed description given herein with respect to the figures is for explanatory purposes only and should not be construed as limiting.

FIG. 1 illustrates a perspective view of an exemplary negative pressure wound therapy system 100 (or “wound therapy system 100” or “system 100”) in accordance with one or more embodiments. The wound therapy system 100 may include a hub 108, a pressure engine 104, tubing 106, and/or dressing(s) 110. For example, as shown in FIG. 1, the wound therapy system 100 includes three distinct dressings 110 to cover and seal three distinct wound sites on the skin 102, although it will be appreciated that any number of dressings 110 may be provided as desired to suit a particular application. Each of the dressings 110 may be attached (e.g., removably) to a respective tubing 106. Each tubing 106 may, in turn, be respectively attached (e.g., removably) to the hub 108, such that the tubing 106 extends between and interconnects the dressing(s) 110 and the hub 108. In this way, in some embodiments, the hub 108 may be aspirationally attached to the dressing(s) 110 via tubing(s) 106. The hub 108 may also be aspirationally attached to a pressure engine 104 that, when activated (e.g., by attachment to the hub 108), may automatically draw pressure from the areas between each dressing 110 and their respective wound sites, thereby creating reduced pressure areas over the wound sites and aspirating fluid away from each dressing 110. Put another way, in embodiments, upon attachment of the pressure engine 104 to the hub 108, the pressure engine 104 may effectively create a vacuum such that fluid is automatically aspirated away from the dressing(s) 110 via a fluid channel 305 extending from the dressing(s) 110, through the hub 108, to the pressure engine 104. As may be appreciated by those skilled in the art, the final sub-atmospheric pressure achieved under the sealed volume contained by the dressing does not necessarily equal that of the sub-atmospheric pressure in the internal chamber of the pressure engine prior to activation. A distribution of sub-atmospheric pressure occurs after activation, resulting in a steady state sub-atmospheric pressure across the entire system (e.g., the dressing, tubing (if any), the hub, and the pressure engine once aspirationally attached to one another). The steady state sub-atmospheric pressure is correlated, but not necessarily equal, to the original sub-atmospheric pressure in the chamber. The correlation is related to mathematical expressions of system volume, external atmospheric pressure at time of use, and internal chamber atmospheric pressure prior to activation.

Although the system 100 is shown as having a single hub 108, a single pressure engine 104, three pieces of tubing 106, and three dressings 110, it should be understood that not all of the depicted components may be used in all embodiments, and one or more embodiments may include more, less, the same, or different components than those shown in the figures. Variations in the arrangement and type of the components may be made without departing from the spirit or scope of the claims as set forth herein. Any number of each component can be used as desired to suit a particular application. The number of tubings 106 may, in embodiments, generally correspond to the number of dressings 110, such that each dressing 110 is aspirationally attached to the hub 108 by a corresponding tubing 106. For example, in some variations, two pieces of tubing 106 may aspirationally attach two dressings 110 to the hub 108. In some variations, four pieces of tubing 106 may aspirationally attach four dressings 110 to the hub 108. In some variations, five pieces of tubing 106 may aspirationally attach five dressings 110 to the hub 108. In some variations, six pieces of tubing 106 may aspirationally attach six dressings 110 to the hub 108. In some variations, seven pieces of tubing 106 may aspirationally attach seven dressings 110 to the hub 108. In some variations, eight pieces of tubing 106 may aspirationally attach eight dressings 110 to the hub 108. Further yet, in some variations, the system 100 may be devoid of any tubing aspirationally attaching the dressing(s) 110 to the hub 108, as described herein. For example, for certain applications (e.g., surgical incisions on the back or spine, laparoscopic surgery, plastic surgeries involving overlapping tissue), including tubing 106 may be particularly advantageous, whereas, for other applications (e.g., where a compact design is preferred), tubing 106 may be omitted. For example, in certain variations, the hub 108 may be directly attached or integrated to the dressing 110, without any tubing. Additional components, different components, or fewer components may be provided in any particular embodiments as desired to suit a particular application. Furthermore, the shape and dimensions of the components depicted in FIG. 1 are intended to be exemplary and non-limiting.

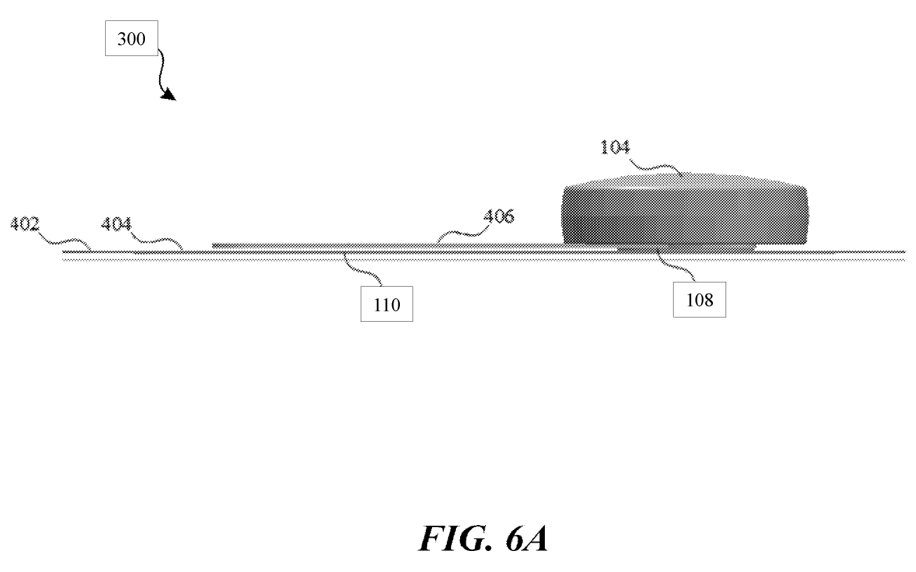

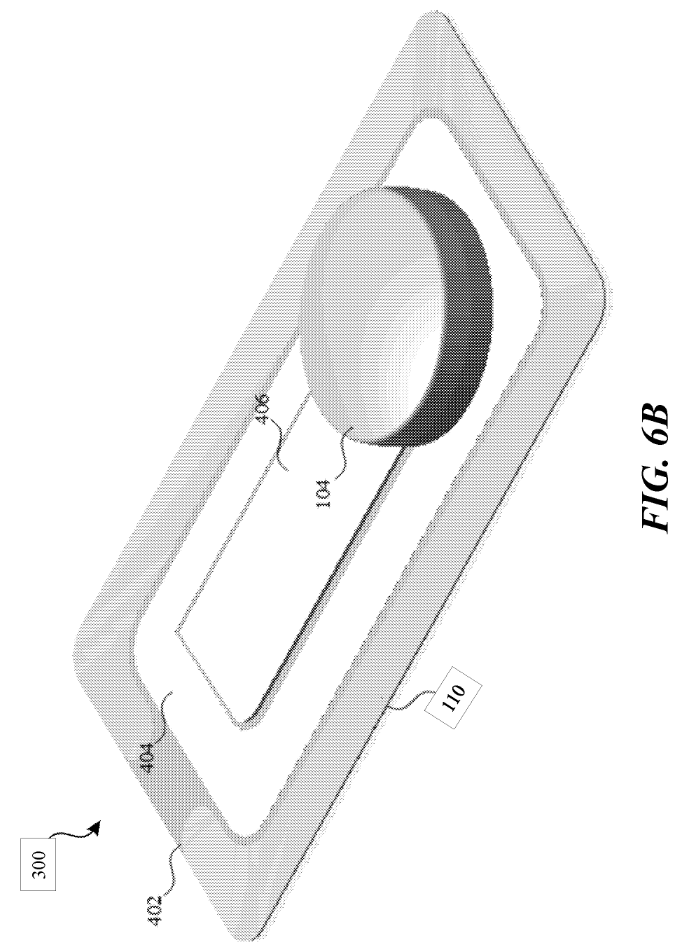

The hub 108 may serve to interface between the dressing(s) 110 and the pressure engine 104. In embodiments employing tubing 106, the hub may serve to interface with the tubing 106 and the pressure engine 104, thereby aspirationally interconnecting the dressing(s) 110 and the pressure engine 104 via the tubing 106 (refer to FIG. 1). In embodiments devoid of any tubing between the dressing 110, the hub 108, and the pressure engine 104, the hub may serve to interface with the dressing 110 and the pressure engine 104, thereby aspirationally interconnecting the dressing(s) 110 and the pressure engine 104 by interfacing directly with both the dressing 110 and the pressure engine 104 (refer to FIG. 6A and FIG. 6B). The hub 108 may be fabricated from a variety of materials, including injection-molded plastics such as acrylonitrile butadiene styrene (ABS), polycarbonate (PC), polypropylene (PP), or the like, including combinations thereof, which may offer a balance of durability, impact resistance, and/or affordability. In some embodiments, the hub 108 may be manufactured from elastomeric materials like silicone, thermoplastic elastomers (TPEs), or the like, including combinations thereof, which may provide flexibility and may be formulated to meet particular biocompatibility and skin-friendliness requirements. In some embodiments, the hub 108 may be formed integrally with the pressure engine 104. In other embodiments, the hub 108 may be formed separately and distinctly from the pressure engine 104 and may be physically attached (e.g., removably) during manufacturing or otherwise. For example, in embodiments, the hub 108 and the pressure engine 104 may be configured to be physically attached to one another, such as directly attached to one another. In some embodiments, the hub 108 and the pressure 104 may be attached to one another with one or more tactile features configured to indicate to a user when the pressure engine 104 is physically attached to the hub 108 and, subsequently, when the pressure engine 104 is aspirationally attached to the hub 108. By way of non-limiting example, a pressure engine interface 308 of the hub 108 and/or a hub interface of the pressure engine 104 may include a tactile or similar features (e.g., levers and ridges) configured to indicate to an user that (i) when one of the pressure engine 104 and the hub 108 are selectively moved (e.g., vertically, rotatingly) relative to the other to a first position (e.g., as a lever passes over a first tactile ridge), the pressure engine 104 is physically attached to the hub 108 but not aspirationally attached to the hub 108 (i.e., the hub 108 and the pressure engine 108 are attached to one another but the internal chamber of the pressure engine 104 is maintained at sub-atmospheric pressure and no fluid is drawn through the hub 108) and (ii) when one of the pressure engine 104 and the hub 108 are selectively moved (e.g., vertically, rotatingly) relative to the other to a second, further position (e.g., as the lever passes over a second tactile ridge), the pressure engine 104 is both physically attached to the hub 108 and aspirationally attached to the hub 108, e.g., with an airtight seal formed between the pressure engine 104 and the hub 108 (i.e., the hub 108 and the pressure engine 108 are attached to one another such that fluid is drawn through the hub 108 toward the pressure engine 104). In some embodiments, when one of the pressure engine 104 and the hub 108 are selectively moved (e.g., vertically, rotatingly) relative to the other, the negative pressure between the pressure engine 104 and the hub 108 may be selectively increased. In some embodiments, the hub 108 and the pressure engine 104 may be physically attached to one another during manufacturing, such that one of the pressure engine 104 and the hub 108 are already pre-selectively moved relative to the other to the first position, requiring the user only to further selectively move one of the pressure engine 104 and the hub 108 to the second position to aspirationally attach the pressure engine 104 to the hub 108 and thereby aspirating fluid away from the dressing(s) 110 via the fluid channel 305 extending from the dressing(s) 110, through the hub 108, to the pressure engine 104.

In terms of design, the hub 108 may be constructed with a range of geometries and features to accommodate different tubing configurations, pressure engine interfaces, and wound site requirements as well as to provide a secure and stable attachment point for the various components of the wound therapy system 100. For example, the hub 108 may be designed with a cylindrical or prismatic shape, with a diameter ranging from 0.5 to 15cm, to accommodate various dressing numbers, sizes, and/or locations. The height of the hub 108 may also be varied, from a few millimeters to several centimeters, allowing for a comfortable fit on the skin 102 or the dressing 110 and to accommodate different tubing lengths and/or different pressure engines. In some embodiments, the surface of the hub 108 may be textured and/or feature a series of ridges or bumps to enhance adhesion to the skin 102 or dressing 110, while also providing a secure grip for the tubing 106 and/or pressure engine 104.

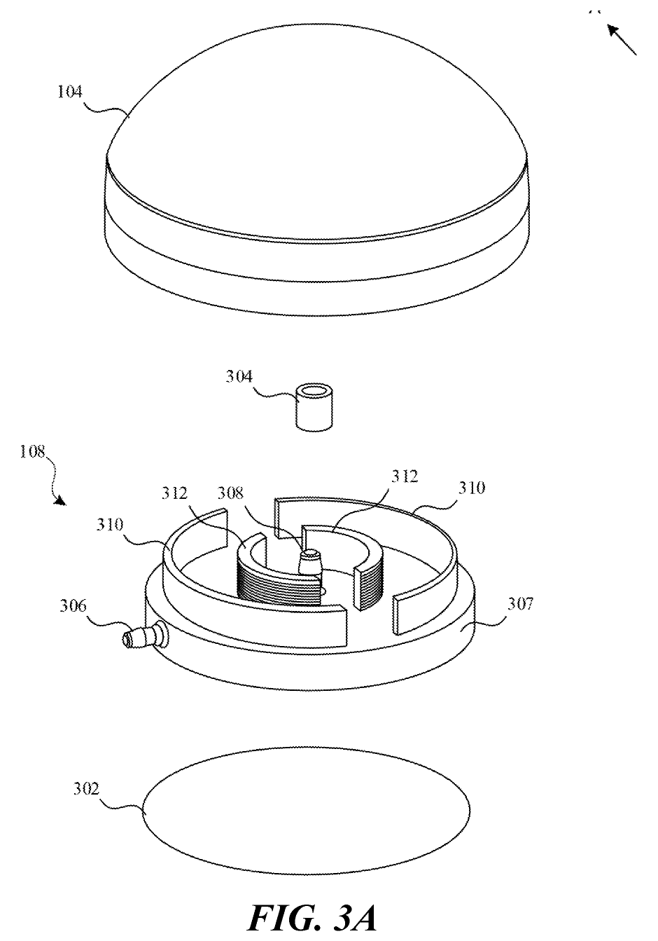

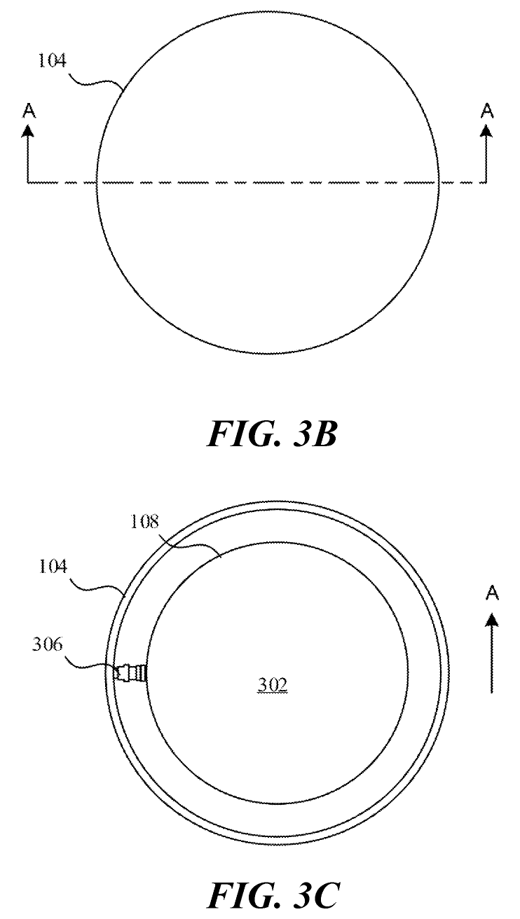

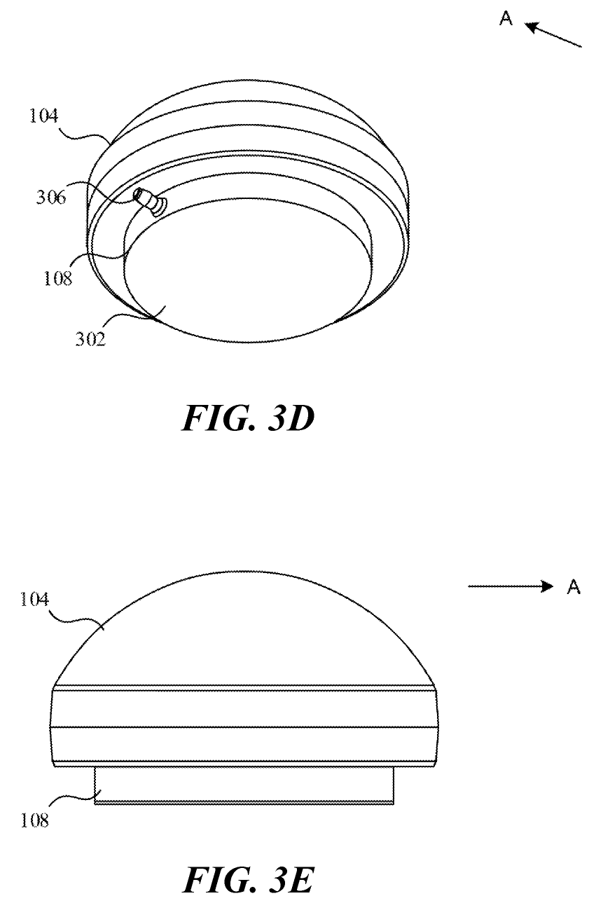

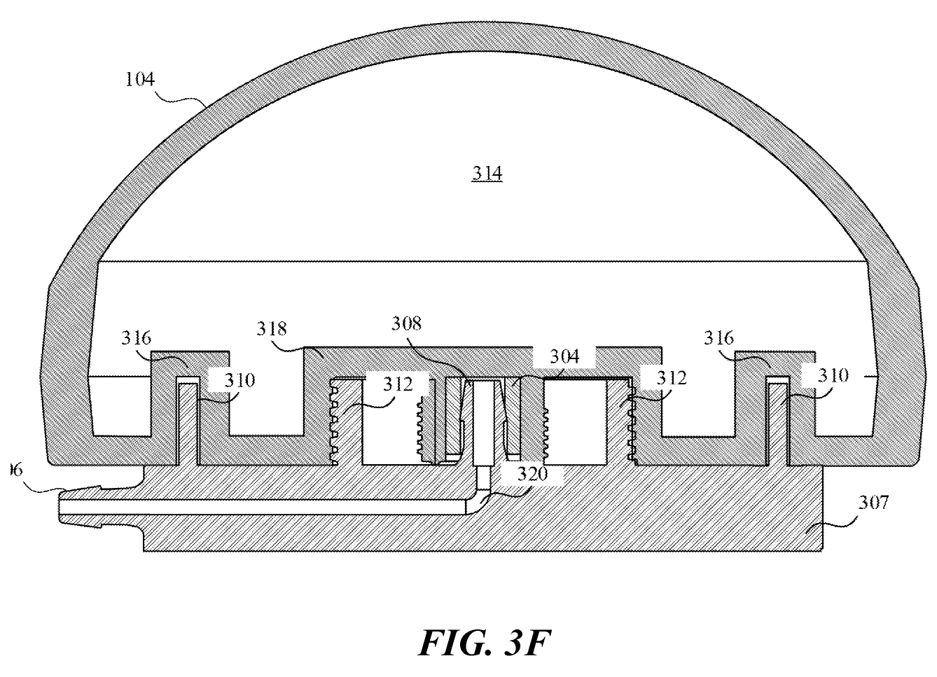

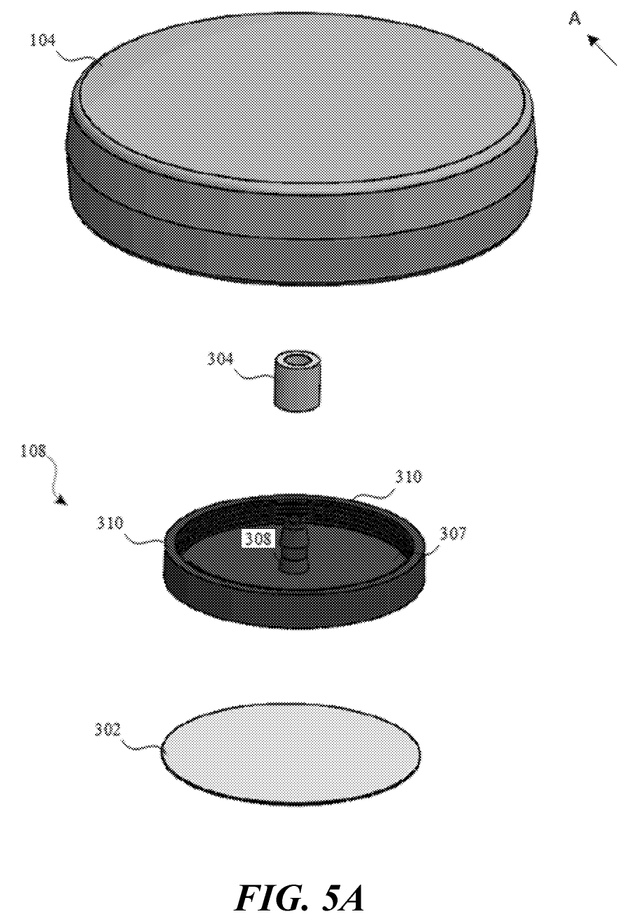

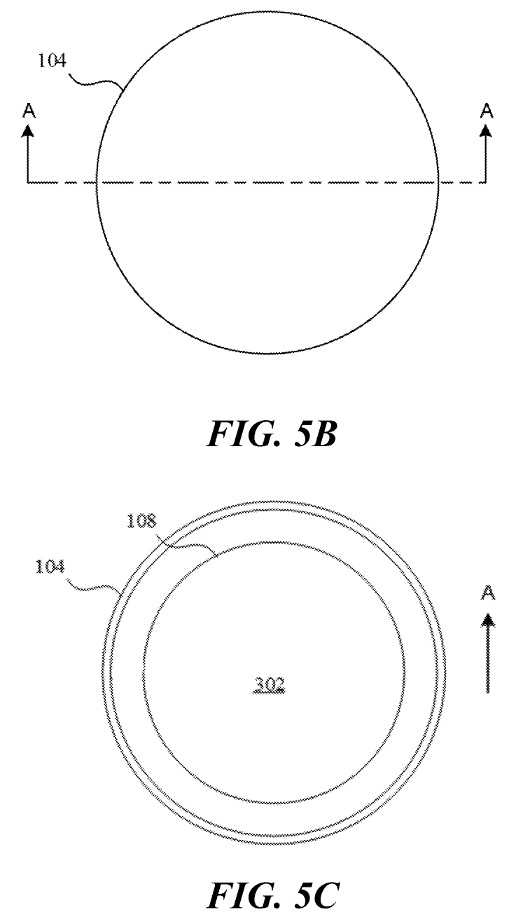

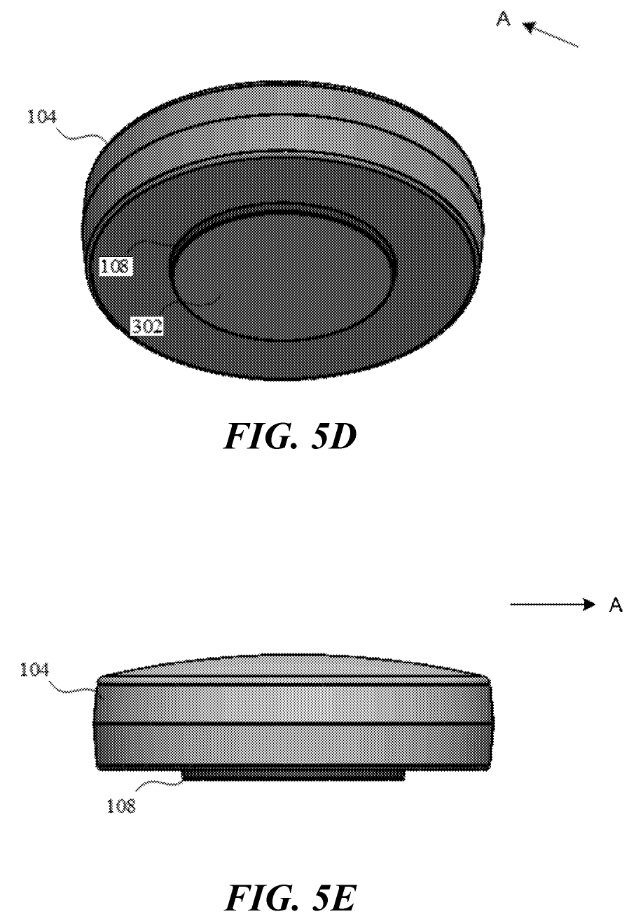

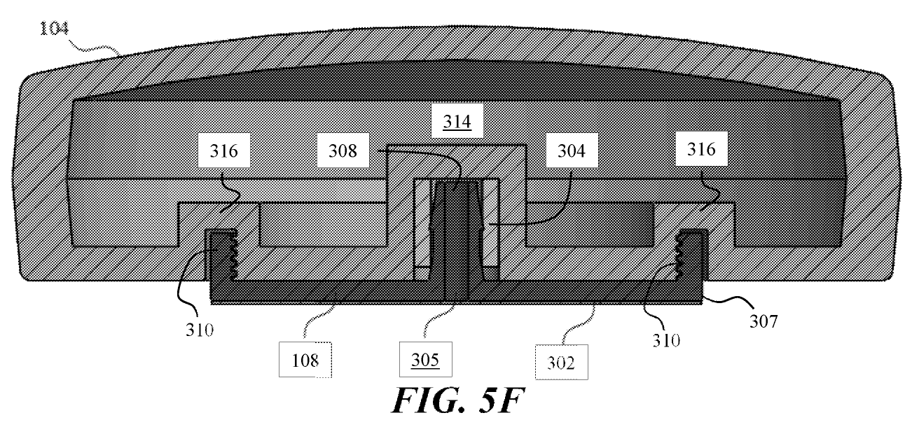

The design of the hub 108 may also incorporate features to facilitate ease of use. In some embodiments, the hub 108 may include a simple snap-on or screw-on interface for attaching the pressure engine 104, or a series of guides or channels to direct the tubing 106 and prevent kinking or occlusion. In some embodiments, the hub 108 may be designed with integrated sensors or indicators (e.g., visual or tactile) to monitor pressure levels, fluid flow, or other relevant parameters, providing real-time feedback to the user and enhancing the overall effectiveness of the wound therapy system 100. In some embodiments, the hub 108 may be constructed with a modular design, allowing for easy replacement or upgrading of individual components, such as the adhesive and/or the pressure engine interface, to extend the lifespan and reduce maintenance costs of the wound therapy system 100. The hub 108 will be described in further detail with respect to FIGS. 3A-3F and 5A-5F.

In some embodiments, the hub 108 (e.g., with the pressure engine 104 physically attached thereto) may be joined with the skin 102 (refer to FIG. 1). For example, the exterior of the hub 108 may include one or more adhesives 302 that may be placed in contact with the skin 102 to secure the hub 108 to the skin 102, such as at a different location than the wound site. The adhesive is further discussed with respect to FIGS. 3A and 5A. In some embodiments, the hub 108 (e.g., with the pressure engine 104 physically attached thereto) may be directly attached to the dressing 110 (e.g., via a dressing interface) (refer to FIGS. 6A and 6B).

The hub 108 may also be aspirationally attached to the pressure engine 104. For example, the hub 108 may include one or more pressure engine interfaces 308 configured to cooperate with one or more corresponding hub interfaces of the pressure engine 104 for aspirationally attaching of the hub 108 to the pressure engine 104 and forming at least a portion of the fluid channel 305. In this way, the aspirational attachment of the hub 108 and the pressure engine 104 may allow the pressure engine 104 to automatically draw, remove, or otherwise aspirate fluid away from other items aspirationally attached to the hub 108, such as a dressing 110. The interface between the hub 108 and the pressure engine 104 is further discussed with respect to FIGS. 3A and 5A. As described herein, in some embodiments, the hub 108 and the pressure engine 104 may describe separate and distinct portions of the same component. For example, the pressure engine 104 may have a pre-evacuated internal chamber 314 for absorbing pressure and a separate hub portion (e.g., formed integrally with the pre-evacuated internal chamber) to attach one or more dressings 110 to the pre-evacuated vacuum portions. In other embodiments, the hub 108 and the pressure engine 104 may describe separate and distinct components that are separately and distinctly manufactured and may be selectively physically attached to one another, during the manufacturing process or otherwise.

The pressure engine 104 may serve to generate a controlled and/or consistent negative pressure (e.g., reduce pressure in and/or remove pressure from one or more dressings 110) to promote wound healing. From a design perspective, the pressure engine 104 may be configured in a variety of ways to achieve the desired pressure level and functionality as desired to suit a particular application. In some embodiments, the pressure engine 104 may have a cylindrical or spherical shape, with a volume ranging from 5cc to 150cc and a diameter of less than 15cm. Such shape may allow for a compact and lightweight design, while also providing a sufficient volume to generate the desired pressure level.

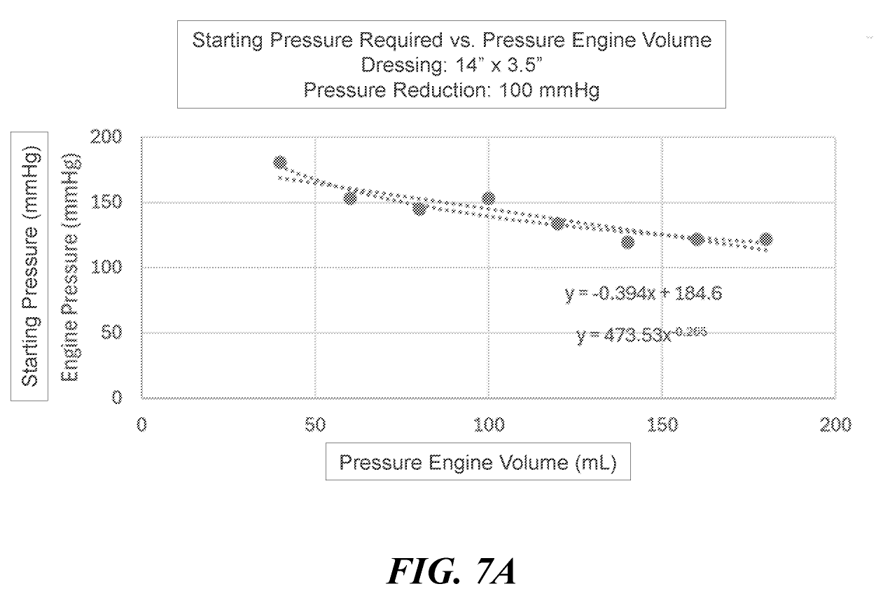

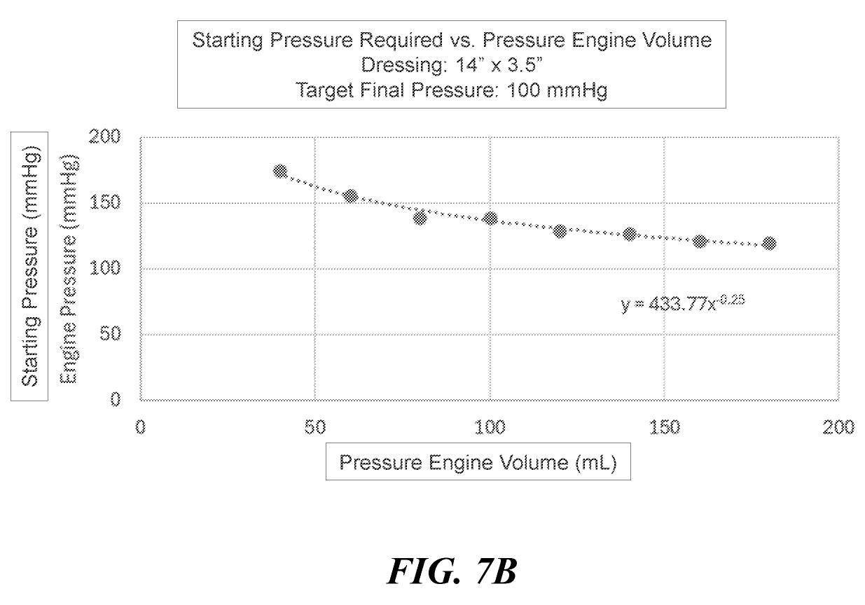

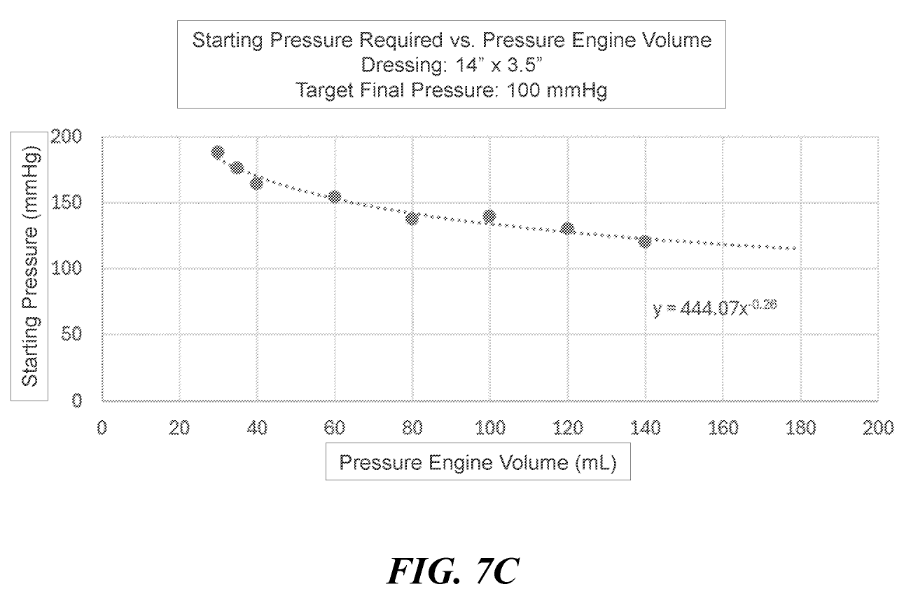

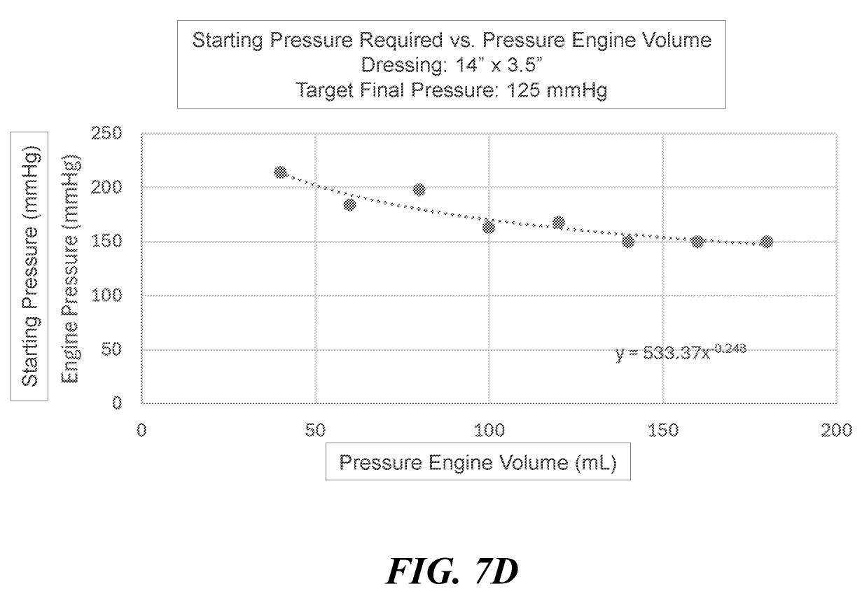

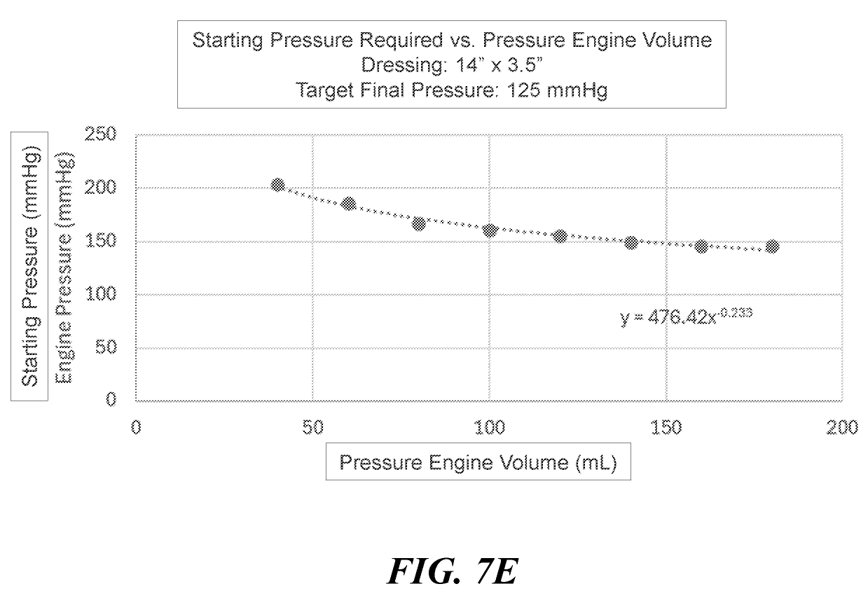

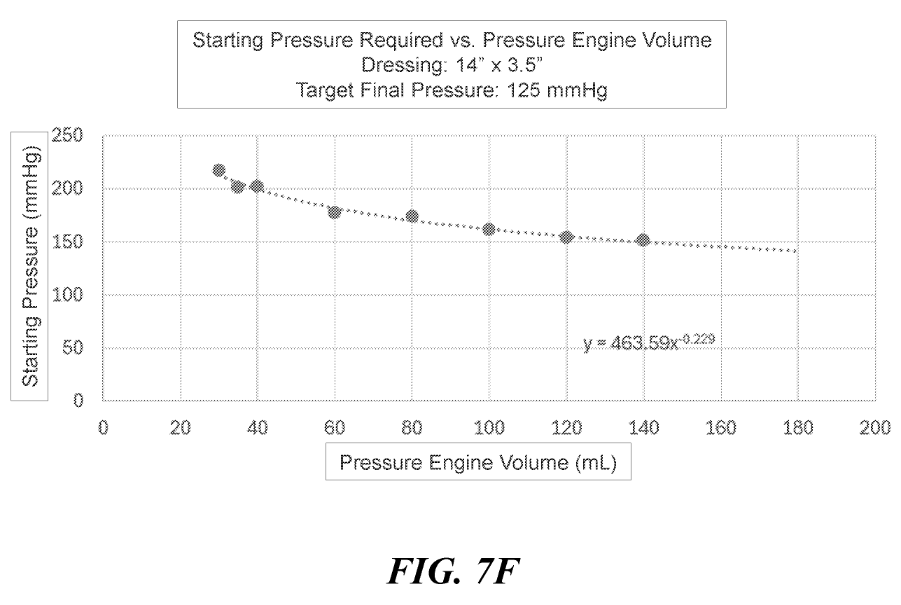

As graphically illustrated in FIGS. 7A-7F, it was found via multiple, consistent tests that for a 14” x 3.5” dressing, that a pressure engine, with a range of volumes from 30mL, to 180mL could produce a therapeutic negative pressure, within the sealed volume contained by the dressing, of 100mmHg relative to the atmospheric pressure at the site of the testing. The starting pressure, relative to the atmospheric pressure during the testing, in the pressure engines were -190mmHg for the 30mL volume and -116mmHg for the 180mL volume. The tests confirmed the efficacy of reducing pressure within a given dressing by employing a pressure engine with a relatively compact design, and the developed numerical system model accurately predicted the behavior of the system with absolute errors of less than 3% in the pressure ranges of interest. The data supports that the system may be designed with a pressure engine volume of 20mL (or smaller) for use with a standard dressing (6” x 1.5” wound pad size). In aspects, the pressure engine may be pre-evacuated to any sub-atmospheric pressure as desired to suit a particular application. For example, in some variations, the pressure engine may be pre-evacuated to from 0mmHg to -400mmHg. For example, in some variations, the pressure engine may be pre-evacuated to from -25mmHg to -175mmHg. For example, in some variations, the pressure engine may be pre-evacuated to from -50mmHg to -150mmHg. For example, in some variations, the pressure engine may be pre-evacuated to from -75mmHG to -125mmHg. For example, in some variations, the pressure engine may be pre-evacuated to -100mmHg. In other variations, sub-atmospheric pressure in the pressure engine can be achieved by techniques other than pre-evacuation, as described herein. By way of non-limiting example, can be formed (e.g., sealed) in a controlled pressure environment, formed (e.g., sealed) in one environment and moved to another environment, or the like, as described herein.

The design of the pressure engine 104 may also incorporate a variety of features to enhance its functionality and performance. For example, the pressure engine 104 may include one or more internal baffles or vanes to help distribute the pressure and/or prevent pressure fluctuations. The pressure engine 104 may also or alternatively include one or more ports or valves to allow for the introduction of additional gases or fluids for pressure relief or adjustment. The pressure engine 104 may also or alternatively include one or more valves, membranes, and/or other flow control devices to regulate the pressure level and/or fluid flow rate. The pressure engine 104 may also or alternatively include one or more internal vacuum chambers, each with its own pressure level and/or characteristics. For example, a single-chamber design may provide a consistent and controlled pressure level, while a multi-chamber design may offer more flexibility and/or adaptability to different wound and/or dressing 110 types and/or sizes.

The pressure engine 104 may be a modular or a cartridge-based design. The pressure engine 104 may be an interchangeable component, allowing for easy replacement and maintenance of the overall wound therapy system 100. A modular or cartridge-based design may enable the use of different pressure levels and/or flow rates, simply by swapping out and/or supplementing different pressure engines with the pressure engine 104. In some embodiments, the pressure engine 104 may be a single-use device, pre-evacuated to a sub-atmospheric pressure designed to be used once (e.g., during a single aspiration process) and then discarded. Pressure within a dressing 110 may be further reduced by the application of one or more subsequent pressure engines. Alternatively, the pressure engine 104 may be designed as a reusable device, with a pressure level that can be adjusted and/or recalibrated as desired and restored after each use. In some embodiments, the pressure engine 104 may be integrated with other components of the negative pressure wound therapy system 100, such as the hub 108, dressing 110, and/or tubing 106, to create a more compact and user-friendly design.

The design of the pressure engine 104 may also include different types of activation components to open the pressure engine 104 and cause the pressure engine 104 to aspirate fluid from a target area, such as the dressing 110 (e.g., causing fluid flow from the dressing 110 to the pressure engine 104). In embodiments, one of the pressure engine 104 and the hub 108 may include a breakable membrane or seal that may be designed to be activated (i.e., broken to allow fluid flow therethrough) by an engagement feature (e.g., screw-on or snap-on interface) included on the other of the pressure engine 104 and the hub 108. In other embodiments, one of the pressure engine 104 and the hub 108 may include a valve that may be opened or closed by an engagement feature (e.g., interface) included on the other of the pressure engine 104 and the hub 108. In this way, upon puncturing of the membrane or seal or opening of the valve as the pressure engine 104 is both physically attached and aspirationally attached to the hub 108, the pressure engine 104 may automatically aspirate fluid away from the dressing in response thereto. The membrane or seal or valve may, in embodiments, be configured to prevent the leakage of air from the pressure engine when the pressure engine 104 is not connected to (e.g., removed from) the hub 108 (e.g., prior to the pressure engine 104 being both physically attached and aspirationally attached to the hub 108). In embodiments, the pressure engine 104 may include a quick-release mechanism, providing a reliable attachment to the hub 108 while also allowing for easy detachment and maintenance. The pressure engine 104 may also include one or more sensors or indicators to provide feedback on the pressure level or system performance.

From a materials perspective, the pressure engine 104 may be fabricated from a variety of biocompatible and durable materials, including ABS plastic, medical polymer equivalents, metallic alloys, and/or other materials that meet medical device standards, including combinations thereof. In the case of a plastic pressure engine, the interior and/or exterior surfaces of the pressure engine may comprise (e.g., be coated with) a metallic film to reduce or eliminate diffusion of gas from the outside world into the low-pressure environment of the sealed inner chamber. The metallic film may be made of non-allergic medical metal, such as aluminum, and can have a thickness ranging from 50 Angstroms to 25 microns. The metallic film may be deposited on the polymer chamber using epitaxy, sputtering, evaporation, or other similar methods. The design and configuration of the pressure engine 104 may account for factors such as sterilization, cleaning, and/or reuse, so that the system 100 may be safely and effectively used in a clinical setting. The pressure engine 104 may be fabricated using a variety of techniques such as injection molding, 3D printing, or computer numerical control (CNC) machining. In embodiments, the pressure engine 104 may include two clam shells plastic molded and/or sealed (e.g., via ultrasonic welding) together to form the pressure engine.

In some embodiments, the hub 108 may be joined with tubing 106. For example, the hub 108 may include one or more tubing interfaces that may be aspirationally attached with one or more corresponding pieces of tubing 106, which are aspirationally attached to or integrated with a corresponding dressing 110. The aspirational coupling of the tubing 106 and dressing 110 allows the pressure engine 104 to draw, remove, or otherwise aspirate fluid away from the dressing 110 via the via the fluid channel 305 extending from the dressing 110, through the hub 108, to the pressure engine 104. The interface is further discussed with respect to FIGS. 3A and 5A.

In some embodiments, the tubing 106 may be configured to aspirationally attach the dressing(s) 110 to the pressure engine 104 (e.g., via the hub 108). The tubing 106 may be fabricated from a variety of flexible materials that may be suitable for medical applications, such as polyethylene, polyvinyl chloride (PVC), polyurethane, silicone, or the like, including combinations thereof. The tubing 106 may be fabricated using a variety of techniques such as extrusion, injection molding, and/or other types of plastic processing depending on the specific design requirements, material selection, and/or production volume. The tubing 106 may be fabricated in a variety of particular colors or opacities, as desired to suit a particular application or desired visual inspection or use capabilities, such as by fabricating the tubing as clear, opaque, or as one or more specific colors.

Due to the proximity of the pressure engine 104 and/or the hub 108 to the dressing 110, the wound therapy system 100 may, in some embodiments, require minimal tubing when compared to other NPWT techniques (refer to FIG. 1). The tubing 106 may be designed to be less than 36 inches long, for example, with a circular cross-section and a diameter ranging from 3/32 inch to 1/4 inch. This range of diameters allows for flexibility in the design of the system 100, enabling the tubing 106 to be used with different wound dressings 110 and pressure engines 104. The length and/or diameter of the tubing 106 may also be optimized to minimize pressure drop and/or maintain consistent pressure delivery to the wound site (e.g., covered by a dressing 110). As described herein, in other embodiments, the hub 108 may be directly attached to the dressing 110 and the pressure engine 110 may be designed so as to be compact enough to obviate the need for any tubing interconnecting the dressing(s) 110 and the hub 108 (refer to FIGS. 6A and 6B), as confirmed by testing of various pressure engine volumes.

The tubing 106 may be designed to be straight, coiled, or bent (in one or more areas), depending on the specific requirements of the system 100. Coiled or bent tubing may provide greater flexibility and convenience, while straight tubing can provide a more direct and efficient attachment between the pressure engine 104 and the dressing 110 when employed. The tubing 106 may also be designed with features such as kink-resistance (e.g., by increasing wall thickness and/or including reinforcement, such as braided or spiral reinforcements), which prevents the tubing 106 from becoming occluded or kinked, and/or pressure-monitoring, which allows for real-time monitoring of the pressure level in the tubing 106.

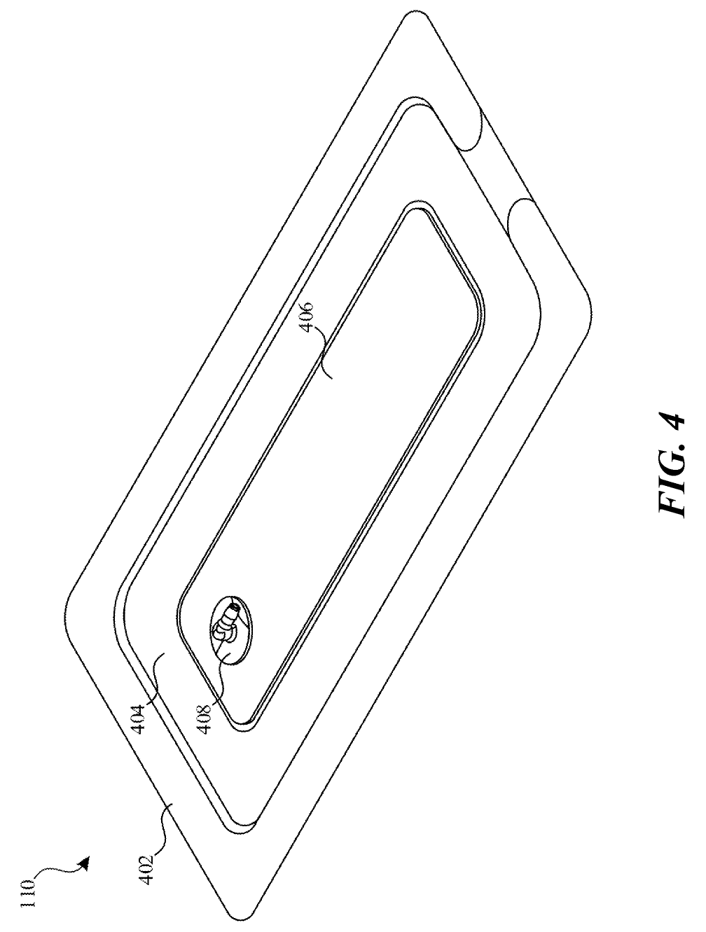

The ends of the tubing 106 may be designed for compatibility with a tubing interface 408 of the dressing 110 (refer to FIG. 4) and/or a tubing interface 306 of the hub 108 (refer to FIG. 3A and FIG. 3F). For example, if the tubing interfaces of the dressing 110 and/or the hub 108 are hose barb connectors, the ends of the tubing 106 may be designed to provide a compatible fit with the hose barb connectors, which provides a secure and airtight connection to the dressing 110 and/or the hub 108. To provide a compatible fit with the hose barb connectors, the ends of the tubing 106 may be designed to be push-fit, snap-fit, or screw fit onto the hose barb connectors. Other possible interface types may include Luer connectors, tube fittings, or the like. In some embodiments, one or more ends of the tubing 106 may be fabricated as part of (e.g., integrally with) the dressing 110, the hub 108, and/or the pressure engine 104. For example, if the tubing 106 is fabricated as part of the dressing 110, the tubing may be removably attached to the hub 108, thus simplifying the application process without sacrificing the removability of the tubing from at least one of the components interconnected by such tubing.

The tubing 106 may also be configured to withstand the negative pressure generated by the pressure engine 104. For example, the tubing 106 may be designed to resist pressures up to -400mmHg relative to atmospheric pressure, which may be a differential pressure sufficient for NPWT applications. The pressure resistance of the tubing 106 may be achieved through a material selection, wall thickness, and/or reinforcement, such as braiding or coiling. For example, to provide pressure resistance up to -400mmHg, the tubing 106 may be polyethylene tubing with a wall thickness of 0.02-0.03 inches or silicone tubing with a wall thickness of 0.050-0.125 inches. In embodiments, the tubing 106 may be cut to a length as desired to suit a particular application, such as to adjust specific application needs as desired.

Each piece of tubing 106 may be attached to one or more dressings 110. The dressing 110 may be responsible for covering and protecting the wound site while facilitating the transfer of negative pressure. The dressing 110 may be fabricated from a variety of materials, including films, foams, gauzes, or the like, including combinations thereof. In embodiments, the dressing 110 may be joined with the skin 102. For example, the exterior of the dressing 110 may include one or more adhesives that may be placed in contact with the skin 102 to secure the dressing 110 to and create an airtight seal with the skin 102 (e.g., via a gasket or similar design feature, as described herein). In embodiments, the dressing 110 may be sterile. The dressing is further discussed with respect to FIGS. 4 and 6A and 6B.

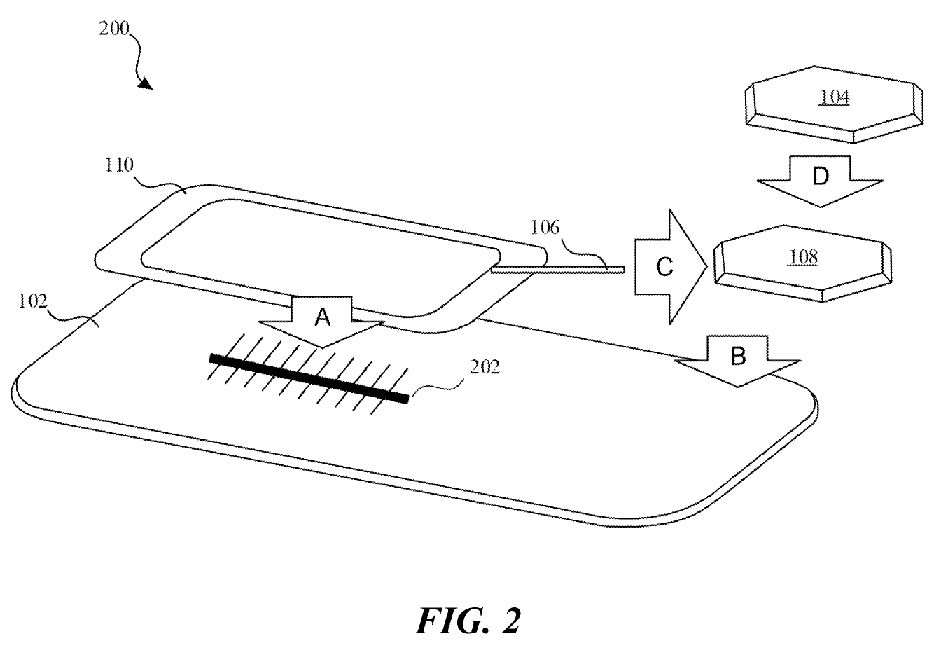

FIG. 2 illustrates an application of an exemplary wound therapy system 100 in accordance with one or more embodiments. Like system 100, system 200 may include a dressing 110, a tubing 106, a pressure engine 104, and a hub 108, the features and functionality of which may be identical to as described in connection with FIG. 1 using the same or similar reference numerals for the same features and/or functionality, except as otherwise described below.

At arrow A, applying the wound therapy system 100 may first include preparing the dressing 110. Preparing the dressing 110 may include selecting a dressing 110 with a size and/or shape that adequately covers the wound site 202. A dressing 110 may adequately cover a wound site 202, for example, if the dressing 110 can cover the entire wound site 202 and create an airtight seal (e.g., via the adhesive and/or gasket of the dressing 110) around the entire wound site 202. Preparing the dressing 110 may also include removing any films, liners, or other materials that preserve the dressing 110, the adhesive of the dressing 110, and/or the cleanliness of the dressing 110. After the dressing 110 is prepared, the dressing 110 may be positioned on (e.g., over) the wound site 202 such that the dressing 110 is attached to the skin 102 surround the wound site (e.g., via an adhesive) and completely covers the wound site 202 and creates an airtight seal with the skin 102 surrounding the wound site (e.g., via a gasket).

At arrow B, applying the wound therapy system 100 may also include preparing the hub 108. Preparing the hub 108 may include selecting a hub 108 that can accommodate the number of dressings 110 being applied, if there is more than one dressing 110 to be applied. In some embodiments, the hub 108 may accommodate more dressings 110 than may be used. For example, as shown in FIG. 2, the hub 108 may accommodate up to four dressings 110, although only one is used. Preparing the hub 108 may include removing any films, liners, or other materials that preserve the hub 108, the adhesive of the hub 108, and/or the cleanliness of the hub 108. In some embodiments, after the hub 108 is prepared, the hub 108 may be placed nearby the dressing 110 (refer to FIG. 1). For example, the hub 108 may be placed within 6 inches of the dressing 110 or a distance corresponding to the length of the tubing 106. If the hub 108 includes more tubing interfaces than dressings 110 and/or pieces of tubing 106 to be used, then the unused tubing interfaces may be sealed (e.g., with a sealing cap or a built-in valve to prevent the transfer of fluid via the unused tubing interfaces). In other embodiments, after the hub 108 is prepared, the hub 108 may be placed on the dressing 110, such as by being directly attached to the dressing 110 (e.g., via a dressing interface of the hub and/or a hub interface of the dressing) (refer to FIGS. 6A and 6B).

At arrow C, when tubing 106 is employed, the tubing 106 may be aspirationally attached to the dressing 110 and the hub 108. The attachment may be such that the tubing 106 allows the communication (e.g., removal, displacement, or otherwise aspiration) of a fluid between the area covered by the dressing 110 and a pressure engine 104 to be attached to the hub 108. The tubing 106 may be inserted, fitted, or otherwise removably attached to a tubing interface 408 of the dressing 110 (e.g., a hose barb) and a tubing interface 306 of the hub 108 (e.g., a hose barb). In some embodiments, the tubing 106 may be integrated or otherwise pre-attached to the dressing 110, in which case the tubing 106 may be removably attached to the hub 108. In other embodiments, the tubing 106 may be integrated or otherwise pre-attached to the hub 108, in which case the tubing 106 may be removably attached to the dressing 110. In some embodiments, the tubing 106 may be integrated or otherwise pre-attached to both the dressing 110 and the hub 108, in which case neither the dressing 110 nor the hub 108 may include a tubing interface for removably attaching the tubing 106.

At arrow D, the pressure engine 104 may be physically attached and/or aspirationally attached to the hub 108. The attachment may be such that the pressure engine 104 is aspirationally attached to the dressing 110 via the hub 108 (e.g., via the tubing 106 or otherwise). The pressure engine 104 may be screwed, snapped, or otherwise removably attached onto a pressure engine interface 308 of the hub 108 (e.g., a hose barb). The pressure engine 104 may include one or more guides for aligning the pressure engine 104 and the hub 108 and/or the hub 108 may have one or more guides for aligning the pressure engine 104 and the hub 108, which may assist in attaching the pressure engine 104 to the hub 108.

Since, in embodiments, the pressure engine 104 is pre-evacuated to a sub-atmospheric pressure, the pressure engine 104 may include an activation mechanism to draw, remove, displace, or otherwise aspirate fluid away from a target source, such as the dressing 110, and/or into the pressure engine 104. In some embodiments, the activation mechanism may be triggered (e.g., automatically) upon attaching the pressure engine 104 to the hub 108. For example, the pressure engine 104 may include a hub interface configured to cooperate with a pressure engine interface 308 of the hub 108. As described herein, in some embodiments, the hub interface of the pressure engine 104 may include a breakable seal or an openable valve configured to be punctured or opened by the pressure engine interface 308 of the hub 108 when the pressure engine 104 is attached to the hub 108, thereby activating the pressure engine 104 and automatically drawing fluid into the pressure engine 104 due to the reduced pressure in the pressure engine 104 relative to the dressing 110. In other embodiments, the structures may be reversed such that the pressure engine interface 308 of the hub 108 may include the breakable seal or valve configured to be punctured or opened by the hub interface of the pressure engine 104 when the pressure engine 104 is attached to the hub 108. As another example, the pressure engine 104 and/or the hub 108 may include a button, switch, or other tactile activation mechanism that, when engaged (e.g., after physically attaching the pressure engine 104 to the hub 108), may activate the pressure engine 104 (e.g., by causing the seal to break or the valve to open), thereby causing fluid to be drawn into the pressure engine 104 due to the reduced pressure in the pressure engine 104 relative to the dressing 110.

Once the system 100 is applied and at least some of the pressure between the dressing 110 and the skin 102 is removed by the pressure engine 104, a pressure equilibrium will be established between the dressing 110 and the pressure engine 104, and the system 100 may remain in place for an amount of time as desired to suit a particular application (e.g., up to seven days without significant loss of negative pressure). In some embodiments, the pressure engine 104 may be removed (e.g., aspirationally detached from the hub 108) after a desired amount of time and/or after aspirating a desired amount of fluid away from the dressing 110 using the pressure engine 104 and replaced with one or more other pressure engines to remove additional pressure from the area covered by the dressing 110 (e.g., by aspirationally attaching the one or more other pressure engines to the hub 108 for a desired amount of time and/or to aspirate a desired amount of fluid away from the dressing 110 using the one or more other pressure engines). The hub 108 may include one or more valves to prevent leaks and allow for the removal and/or replacement of additional pressure engines.

FIG. 3A illustrates an exploded view of an exemplary pressure engine 104 and hub 108 in accordance with one or more embodiments.

The hub 108 may be secured to the skin 102 via a layer of adhesive 302. The adhesive may be designed to provide a secure and comfortable seal on the skin 102. The adhesive 302 may include materials such as polyacrylate adhesives, hydrocolloid adhesives, silicone adhesives, or the like, including combinations thereof. The adhesive 302 may be applied to the entire contact surface of the hub 108 or limited to specific areas, depending on the desired level of adhesion and skin-friendliness. In some embodiments, the adhesive 302 may be coated onto the bottom of the hub 108. In some embodiments, the adhesive 302 may be a double-sided adhesive that may be applied to the hub 108 and the skin 102 so that the adhesive 302 may be cut or otherwise formed into a desired shape.

The hub 108 may include a tubing interface 306 and/or a pressure engine interface 308. As shown in FIG. 3A, the tubing interface 306 and/or the pressure engine interface 308 may be hose barbs protruding outwardly from the hub 108. In some embodiments, the tubing interface 306 and/or the pressure engine interface 308 may be constructed from ABS plastic molded as part of the hub 108. In some embodiments, the tubing interface 306 and/or the pressure engine interface 308 may be constructed as a separate insert piece attached to the hub 108. In some embodiments, the tubing interface 306 and/or the pressure engine interface 308 may be constructed from different materials, such as ABS plastic or metal. For example, the tubing interface 306 may be molded as part of the hub 108, whereas the pressure engine interface 308 may be a separate metallic piece attached to the hub 108, which may be more suitable for piercing any seals that may be included in the hub interface of the pressure engine 104.

The hub 108 may include the same, a lesser number, or a greater number of tubing interfaces 306 than pressure engine interfaces 308, and the tubing interface(s) 306 may attach with the pressure engine interface(s) 308 inside the hub 108. The interior of the hub 108 may be defined by a wall 307 that encloses one or more sides, a top, and/or a bottom of the hub 108.

The hub 108 may also include one or more guides to help align and attach and secure the pressure engine 104 to the hub 108. The guides may be in the form of walls, groves, pins, or the like, including combinations thereof. As shown in FIG. 3A, the hub 108 may include inner guide walls 312. The inner guide walls 312 may protrude from an upper surface of the hub 108 and may be received by corresponding groves in the pressure engine 104 (e.g., as shown in FIG. 3F). The inner guide walls 312 may reduce or prevent lateral and/or longitudinal movement of the pressure engine 104 on the hub 108 to prevent movement that causes pressure loss and/or fluid leakage at the pressure engine interface 308 of the hub 108 when the pressure engine 104 is attached to the hub 108. The inner guide walls 312 may include threading, flanges, slots, or the like, including combinations thereof to align the pressure engine 104 with the hub 108 and secure the pressure engine 104 to the hub 108 and reduce or prevent vertical movement of the pressure engine 104 relative to the hub 108. As shown in FIG. 3A, the hub 108 may also include outer guide walls 310. The outer guide walls 310 may be placed at a position further from the center of the hub 108 than the inner guide walls 312. The outer guide walls 310 may serve a similar purpose as the inner guide walls 312 and thus may include similar features as the inner guide walls 312. For example, as shown in FIG. 3A, the outer guide walls 310 may also protrude from the upper surface of the hub 108, helping to reduce or prevent lateral and/or longitudinal movement of the pressure engine 104 relative to the hub 108. As will be appreciated by those skilled in the art, such structures could be reversed such that the pressure engine 104 includes inner and/or outer guide walls that may protrude from a lower surface of the pressure engine 104 and may be received by corresponding groves in the hub 108.

The pressure engine 104 may be placed on top of the hub 108 and secured to the hub 108 by the pressure engine interface 308 (e.g., including cooperation between the pressure engine interface 308 of the hub and the hub interface of the pressure engine 104), the inner guide walls 312, and/or the outer guide walls 310. The pressure engine 104 may have corresponding grooves or receptacles to receive the pressure engine interface 308, the inner guide walls 312, and/or the outer guide walls 310. In some embodiments, the hub 108 may instead have one or more grooves or receptacles to receive one or more corresponding walls and/or interfaces of the pressure engine 104.

As shown in FIG. 3A, the pressure engine 104 may be screwed on to or otherwise engaged with the hub 108 via threads. The pressure engine interface 308 may narrow or otherwise be configured to pierce a seal or operate (e.g., push) a valve that opens the pre-evacuated internal chamber 314 of the pressure engine 104, causing the pressure engine 104 to absorb fluid from target source(s) attached to the tubing interface 306, such as a tubing 106 which is attached to the dressing 110, thereby drawing pressure away from the target source(s) (e.g., the dressing 110).

In some embodiments, the pressure engine 104 and/or the hub 108 may include a gasket 304 to further reduce or prevent movement of the pressure engine 104 relative to the hub 108 and/or reduce or prevent any fluid leaks between the pressure engine 104 and the hub 108 (e.g., at the pressure engine interface 308). The gasket 304 may be substantially the same height and/or diameter as the pressure engine interface 308, and the gasket 304 may be a pliable material (e.g., rubber) to fill gaps that may form between the pressure engine interface 308 and the pressure engine 104.

It should be understood that the size, shape, and features presented in FIG. 3A are merely exemplary and non-limiting. For example, the pressure engine 104 and hub 108 may be prisms rather than cylinders. In some embodiments, the pressure engine 104 and hub 108 may even include differing sizes, shapes, and/or features as desired to suit a particular application. For example, the hub 108 may be a cylinder, and the pressure engine 104 may be a cube with a base surface smaller than the top surface of the hub 108.

FIG. 3B illustrates a top view of the exemplary pressure engine 104 and hub 108 of FIG. 3A in accordance with one or more embodiments. As shown in FIG. 3B, the pressure engine 104 may be the same or larger diameter than the hub 108, thus covering or encompassing the hub 108. In some embodiments, the hub 108 may be designed to accommodate pressure engines of various shapes and/or sizes. Accordingly, in some embodiments, the hub 108 may be visible from the top view. In other embodiments, the hub 108 may be at least partially or completely non-visible and occluded from the top view by being at least partially or completely covered or encompassed by the pressure engine 104.

FIG. 3C illustrates a bottom view of the exemplary pressure engine 104 and hub 108 of FIG. 3A in accordance with one or more embodiments. As shown in FIG. 3B, the hub 108 may be a smaller diameter than the pressure engine 104, and thus the pressure engine 104 may be visible from the bottom view. In some embodiments, the hub 108 may be designed to accommodate pressure engines of various shapes and/or sizes. Accordingly, in some embodiments, the hub 108 may be substantially the same diameter as the pressure engine 104, such that the pressure engine 104 may be at least partially or completely non-visible from the bottom view and occluded from the bottom view by being at least partially or completely occluded by the hub 108.

FIG. 3D illustrates a perspective view of the exemplary pressure engine 104 and hub 108 of FIG. 3A in accordance with one or more embodiments. As shown in FIG. 3D, the pressure engine 104 may be physically attached to the hub 108, which may result in disk-shaped device defined by the combination of the hub 108 and the pressure engine 104. The hub 108 may include one or more attachment points (e.g., the tubing interface 306) for attaching various components, such as tubing 106 and/or sensors, to the hub 108.

FIG. 3E illustrates a side view of the exemplary pressure engine 104 and hub 108 of FIG. 3A in accordance with one or more embodiments. It should be understood that although the pressure engine 104 is shown as being larger than the hub 108, other shapes and sizes of the pressure engine 104 and hub 108 are contemplated as desired to suit a particular application. Thus, in some embodiments, the hub 108 may be larger than the pressure engine 104, for example, to accommodate more attachments such as pressure engines and/or dressings. In other embodiments, the hub 108 may be smaller than or substantially the same size and/or diameter as the pressure engine 104.

FIG. 3F illustrates a side sectional view of the exemplary pressure engine 104 and hub 108 of FIG. 3A in accordance with one or more embodiments. The pressure engine 104 may include one or more walls defining the internal chamber 314. The pressure engine 104 may include a metallic film over at least a portion of an interior surface of the internal chamber 314. Additionally or alternatively, the pressure engine 104 may include a metallic film over at least a portion of an external surface of the pressure engine 104. In variations, the pressure engine 104 may include a metallic film covering substantially all of the internal surface and/or the external surface of the pressure engine 104. The pressure engine 104 may be devoid of an active vacuum source (e.g., electromechanical, mechanical, or chemical); rather, the internal chamber 314 of the pressure engine 104 may be pre-evacuated to a pressure less than atmospheric pressure (i.e., a sub-atmospheric pressure). In some embodiments, to pre-evacuate the internal chamber 314, a vacuum may be applied to the internal chamber 314 during the manufacturing process. For example, during manufacturing, air may be evacuated from the internal chamber 314 of the pressure chamber 104 such that the internal chamber 314 of the pressure engine 104 is at a sub-atmospheric pressure. Thereafter, the internal chamber 314 of the pressure engine 104 may be sealed so as to maintain the internal chamber 314 at the sub-atmospheric pressure. In some embodiments, to continuously evacuate the chamber 314, a vacuum may be applied to the internal chamber 314 during the fluid aspiration process. The vacuum may be generated using a vacuum pump or other type of vacuum source. The vacuum may be applied to the internal chamber 314 through a small opening or port, which may then be sealed once the desired pressure level is achieved. In other embodiments, the pressure engine 104 may be formed in a sub-atmospheric pressure environment (e.g., within a sub-atmospheric device) such that the internal chamber 314 of the pressure engine 104 is at a sub-atmospheric pressure. Additionally or alternatively, the sub-atmospheric pressure environment may contain a custom blend of medical gases to be entrapped in the pressure engine 104, rather than the blend of gases typically found in the atmosphere on the surface of the earth. Thereafter, the internal chamber 314 of the pressure engine 104 may be sealed so as to maintain the internal chamber 314 at the sub-atmospheric pressure (e.g., when the pressure engine 100 is removed from the sub-atmospheric pressure environment). In other embodiments, the pressure engine 104 may be formed in a first environment (e.g., at an altitude of 10,000 feet or more above mean sea level, such as in Leadville, Colorado) such that the internal chamber 314 of the pressure engine 104 is at an internal pressure corresponding to the external atmospheric pressure in the first environment. Thereafter, the internal chamber 314 of the pressure engine 104 may be sealed so as to maintain the internal chamber 314 at the internal pressure, and the pressure engine 104 may be moved to a second environment (e.g., at an altitude of 100 feet or less above mean sea level, such as in Houston, Texas) different from the first environment, such that the internal pressure of the internal chamber 314 of the pressure engine 104 is sub-atmospheric relative to the external atmospheric pressure in the second environment (e.g., after the pressure engine 100 is moved from the first environment to the second environment). In the same or alternative embodiments, during manufacturing of the pressure engine 104, the temperature of the ambient air could be increased, thereby reducing the density of the air to be captured within the internal chamber 314 of the pressure engine 104. As a result, upon sealing of the engine 104, the air captured within the internal chamber 314 will cool, resulting in a lower absolute pressure within the internal chamber 314 of the pressure engine 104.

Once the internal chamber 314 is pre-evacuated, the internal chamber 314 may be sealed. Once the pressure engine 104 with the sealed internal chamber 314 is attached to the hub 108 (and tubing 106, when employed), the hub 108 may activate the pressure engine 104, causing the seal of the hub 108 or the internal chamber 314 of the pressure engine 104 to be broken (e.g., valve to be opened), thereby allowing the negative pressure of the internal chamber 314 to be communicated to the dressing 110 to draw, remove, or otherwise aspirate fluid away from the dressing 110 and toward the pressure engine 104.

The hub 108 may include one or more walls (e.g., wall 307) that define an interior of the hub 108. As shown in FIG. 3F, the interior of the hub 108 may be solid. In some embodiments, the interior of the hub 108 may be hollow. The interior of the hub 108 may include one or more fluid channels 320 between the one or more tubing interfaces 306 and the one or more pressure engine interfaces 308. For example, the multiple tubing interfaces may be attached to a single pressure engine interface via the fluid channel 320 so that the pressure engine 104 may evenly remove fluid from the dressing(s) 110 attached to the multiple tubing interfaces to reduce pressure from the dressing(s) 110.

On the top of the hub 108 may be one or more walls for guiding the placement of and/or securing the pressure engine 104. The hub 108 may include inner guide walls 312 and/or outer guide walls 310, which may help align the hub 108 and the pressure engine 104 and reduce or prevent lateral and/or longitudinal movement of the pressure engine 104 once attached to the hub 108. The pressure engine 104 may include one or more guide receptacles for receiving the guide walls of the hub 108. As shown in FIG. 3F, the pressure engine 104 may include a set of inner guide receptacles 318 for receiving the inner guide walls 312. The inner guide receptacle 318 may have a threading corresponding to the threading of the inner guide walls 312. The pressure engine 104 may also include a set of outer guide receptacles 316 for receiving the outer guide walls 310 of the hub 108. As described herein and as will be appreciated by those skilled in the art, such structures could be reversed such that the pressure engine 104 includes inner and/or outer guide walls that may protrude from a lower surface of the pressure engine 104 and may be received by corresponding guide receptacles in the hub 108.

The hub 108 may include one or more tubing interfaces 306. The tubing interface 306 may be a hose barb or other tube fitting that protrudes from the hub 108 (e.g., on the side as shown in FIG. 3D), allowing for easy attachment and detachment of the tubing 106. The tubing interface 306 may include features such as tapered tips, ridges, or textured surfaces to enhance the securement of the tubing 106 and prevent kinking or occlusion. In some embodiments, the hub 108 may also or alternatively incorporate a push-fit or snap-fit connector, which allows the tubing 106 to be simply pushed or snapped onto the hub 108, eliminating the need for additional fittings or adapters.

The hub 108 may include one or more pressure engine interfaces 308. The pressure engine interface 308 may also be hose barb or other fitting that protrudes from the hub 108 (e.g., on the top) providing a secure and reliable attachment, while also facilitating easy attachment and detachment. In some embodiments, the pressure engine interface 308 may include a screw-on or threaded connection, where the pressure engine 104 may be screwed onto the hub 108 using a series of threads or a bayonet-style connector. In some embodiments, the pressure engine interface 308 may include a snap-on or clip-on connection, where the pressure engine 104 may be snapped or clipped onto the hub 108.

The hub 108 may also include various features to enhance the attachment and functionality of the tubing 106 and/or pressure engine 104. For example, the hub 108 may include one or more seals or gaskets (e.g., gasket 304) to provide an airtight connection between the tubing 106 and pressure engine 104, or a valve or check valve to regulate the flow of pressure and prevent backflow. Preventing backflow may also allow the application of multiple pressures engines 104 to further reduce the pressure in the area covered by the dressing 110. For example, one pressure engine may be attached to the hub 108 to reduce the pressure within the dressing 110 by, for example, 100mmHg, and the pressure engine may be removed and replaced with another pressure engine, which may be attached to the hub 108 to further reduce the pressure within the dressing 110 by, for example, another 100 mmHg.

FIG. 4 illustrates a perspective view of an exemplary dressing 110 in accordance with one or more embodiments. The dressing 110 may be a multi-component system designed to maintain airtightness over a wound site and facilitate negative pressure wound therapy.

In embodiments, the dressing 110 may include a gasket 404. The gasket 404 may operate as the part of the dressing 110 that may adhere to the area around the wound site and/or create an airtight seal surrounding the wound site (e.g., wound site 202). The gasket 404 may include a central opening (e.g., may be doughnut-shaped). The gasket 404 may be made in various shapes and sizes, depending on the type and location of the wound. For example, the gasket 404 may be circular, oval, rectangular, or custom-made. If the gasket 404 is circular, the gasket 404 may have a 3-20cm outer diameter, 1-15cm inner diameter, and a 1-5cm width. If the gasket 404 is rectangular, the gasket 404 may have a 3-45cm outer width, 4-90cm outer length, 2-25 inner width, and a 3-70 inner length. If the gasket 404 is square, the gasket 404 may have a 3-17cm outer length and a 1-8cm inner length. The gasket 404 may also be made in a variety of thicknesses, such as ranging from 0.2mm to 5mm, depending on the desired level of cushioning and conformability.

The gasket 404 may be made of a medical polymer that is compatible with the skin and tissue, such as silicone, polyurethane, hydrogel, or the like, including combinations thereof. The gasket 404 may be coated and/or impregnated with other substances, such as antimicrobial agents, growth factors, pain relievers, or the like, including combinations thereof to enhance the healing process and reduce the risk of infection. In embodiments, the dressing 110 may include a drape 402. The gasket 404 may be attached to the drape 402 by various methods, such as adhesive bonding, heat bonding, mechanical fastening, or the like, including combinations thereof. The gasket 404 may have sufficient viscosity and elasticity to conform to the skin surface or other surfaces, but with a surface tension sufficient to keep its shape and prevent spreading. The gasket 404 may also be able to withstand the pressure and vacuum applied by the pressure engine 104, without compromising its integrity or seal.

The drape 402 may be a flexible sheet that covers the entire dressing 110, including the wound site, and provides a sterile barrier to prevent infection. The drape 402 may be made from polymer film that resists fluids. The polymer film can be made of polyethylene, polypropylene, polyester, or other medical polymers, including combinations thereof, and can have a thickness of less than 5mm. The drape 402 may be formed in different shapes and sizes depending on the type and location of the wound. The shape and/or size of the drape 402 may correspond to the shape and/or size of the gasket 404. In some embodiments, the drape 402 may be larger than the gasket 404 (e.g., up to 8cm).

The drape 402 may include a metallic, ceramic, and/or polymeric (e.g., Hydrocolloid – Carbomethylcellulose) film on (e.g., coated) at least one side of the drape 402 and at least inside the area defined by the gasket 404 to reduce the air permeating through the drape 402. Such a drape with a metallic film is described in U.S. Patent Publication No. 2023/0218444 (having inventors in common with the present application), the contents of which are incorporated by reference herein. The metallic film may be made of non-allergic medical metal, such as aluminum, and can have a thickness ranging from 50 Angstroms to 25 microns. The metallic film may be deposited on the polymer film using epitaxy, sputtering, evaporation, or other similar methods.

The drape 402 may be attached to the skin 102 and/or the gasket 404 using an adhesive that is compatible with both the polymer film and the skin 102. The adhesive may cover the surface of the drape 402 that contacts the skin 102 or only a portion of the surface, such as the edges or the corners of the drape 402. The adhesive may have a thickness ranging from 10 microns to 1mm. The adhesive may be a polyacrylate adhesive material or other medical grade adhesive with sufficient adherence to skin. The adhesive may securely hold the drape 402 in place, and the drape 402 may hold the gasket 404 in place, which forms the airtight seal.

The dressing 110 may also include one or more absorptive pads 406 that may contact the wound site and facilitate wound healing. The absorptive pad 406 may be placed on the side of the drape 402 that faces the wound site and that contains the gasket 404 and/or adhesive. The absorptive pad 406 may be made from various materials that can absorb wound exudate, such as woven or nonwoven gauze, open face polyurethane foam, nonwoven super absorbent polymer, hydrophilic compositions, or the like, including combinations thereof. The absorptive pads 406 may also have a wound contact layer that protects the wound bed from sticking to the pads and reduces pain and trauma during dressing removal. The absorptive pad 406 may be shaped and sized to fit the wound dimensions (e.g., to cover the entire wound area). The absorptive pads 406 may benefit wound healing, for example, by absorbing excess fluid, preventing leakage and infection, maintaining a moist wound environment, and/or holding fluid away from wound site.

In some embodiments, at least part of the absorptive pad 406 and/or the drape 402 may include a volume control element to mitigate the shrinkage of the dressing 110 or components thereof (e.g., the absorptive pad 406) while under the effect of reduced pressure. The volume control element may be one or more pieces of moldable medical polymer in a lattice, honeycomb, or other structure. The volume control element may be part of the absorptive pad 406 and/or the drape 402. The volume control element may be the same size as the absorptive pad or smaller so as to help maintain the rigidity of the portion of the dressing 110 over the wound. In some embodiments, volume control may alternatively or additionally be achieved by employing a dressing 110 and/or structural element (e.g., an absorptive pad 406 with a sufficient degree of stiffness) to resist volume change under the dressing under the differential pressure during use. Volume control to mitigate shrinkage of the dressing 110 or components thereof (e.g., the absorptive pad 406) results in the ability to employ a smaller (e.g., less voluminous) pressure engine, which may be particularly advantageous for certain applications.

A tubing interface 408 may be located on the drape 402 and/or the gasket 404. The tubing interface 408 may be positioned to minimize interference with the wound site and/or the wearer’s movement. The tubing interface 408 may be sealed to the drape 402 and/or the gasket 404 using adhesive, heat, and/or other methods. The tubing interface 408 may be a connector that allows for easy attachment and detachment of tubing 106. The tubing interface 408 may be a solid material, such as plastic or metal. The tubing interface 408 may be a hose barb, a quick-release coupling, a threaded fitting, or another form of connector. When the tubing 106 is attached to the dressing 110 (e.g., via the tubing interface 408) and the pressure engine 104, the pressure engine 104 may reduce pressure in and/or remove pressure from the target area, which may be defined by the drape 402, gasket 404, and/or skin 102. The pressure engine 104 may reduce and/or remove pressure by withdrawing a predetermined (e.g., corresponding to the negative pressure pre-evacuated from the chamber 314) amount of fluid (e.g., air) from the target area, creating a NPWT environment. In some embodiments, the tubing interface 408 may include a one-way valve that prevents fluid (e.g., air) from entering the target area when the dressing 110 is detached from the pressure engine 104.

FIGS. 5A-5F illustrate an exemplary hub 108 and pressure engine 104 in accordance with one or more embodiments, the features and functionality of which may be identical to as described in connection with FIGS. 3A-3F using the same or similar reference numerals for the same features and/or functionality, except as otherwise described below. FIGS. 6A and 6B illustrate an application of an exemplary wound therapy system 300 in accordance with one or more embodiments. Similar to system 100 and system 200, system 300 may include a dressing 110, a pressure engine 104, and a hub 108 (e.g., the pressure engine 104 and hub 108 illustrated in FIGS. 5A-5F), the features and functionality of which may be identical to as described in connection with FIGS. 1 and 2 using the same or similar reference numerals for the same features and/or functionality, except as otherwise described below. In particular, system 300 is devoid of any tubing between the dressing 110 and the hub 108; rather, as described herein, the hub 108 is directly attached to the dressing 110, thereby obviating the need for any tubing between the dressing 110 and the hub 108.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to,” “operable to,” and “programmed to” do not imply any particular tangible or intangible modification of a subject, but, rather, are intended to be used interchangeably. In one or more embodiments, a processor configured to monitor and control an operation or a component can also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an embodiment, the embodiment, another embodiment, some embodiments, one or more embodiments, an embodiment, the embodiment, another embodiment, some embodiments, one or more embodiments, a configuration, the configuration, another configuration, some configurations, one or more configurations, the subject technology, the disclosure, other variations thereof and alike are for convenience and do not imply that a disclosure relating to such phrase(s) is essential to the subject technology or that such disclosure applies to all configurations of the subject technology. A disclosure relating to such phrase(s) can apply to all configurations, or one or more configurations. A disclosure relating to such phrase(s) can provide one or more examples. A phrase such as an aspect or some aspects can refer to one or more aspects and vice versa, and this applies similarly to other foregoing phrases.

Unless specified otherwise, any component described herein can include a plurality of such components or only one of such component, as desired to suit a particular application, without departing from the scope or spirit of the present disclosure.

While aspects described with respect to drawings and methods herein may differ, it will be understood by those of skill in the art, on review of the disclosures herein, that all aspects expressed with respect to such methodologies may be implemented using systems disclosed herein, and all aspects described as to systems herein can be performed by methodologies disclosed herein, without departing from the scope or spirit of the innovation. Moreover, steps of methodologies and/or components of systems can be added, removed, consolidated, divided, re-ordered, and the like, as desired to suit a particular application, without departing from the scope or spirit of the present disclosure. Embodiments may also be combined, in whole or in part, without departing from the scope or spirit of the present disclosure.

The term “airtight” is used herein to mean sufficiently resistive to uncontrolled air leakage or air flow. In embodiments, “airtight” may refer to uncontrolled air leakage of not greater than 25% as uncontrolled air leakage or air flow of greater than 25% may result in final pressure at application being suboptimal. The term “airtight seal” is used herein to mean a seal that is sufficiently “airtight” so as not to impair a process, such as to maintain a negative pressure to permit fluid to be drawn, removed, or otherwise displaced from one component to another by or as if by suction.

The term “attached to” is used herein to mean that two components are connected to one another, whether or not the connection involves an intermediate component. The term “directly attached to” is used herein to mean that two components are physically connected to one another adjacent one another, without the connection involving an intermediate component.

The term “aspirationally attached to” is used herein to mean that two components are connected to one another such that fluid may be drawn, removed, or otherwise displaced from one of the components to the other of the components by or as if by suction.

The term “sub-atmospheric pressure” is used herein to mean below external atmospheric pressure (i.e., below 760mmHg at sea level) in the particular environment (e.g., a differential pressure sufficient for NPWT applications). With respect to a sealed pressure engine pre-evacuated to a sub-atmospheric pressure, the result is sub-atmospheric pressure (e.g., vacuum) is created and maintained within the pressure engine, as described herein. With respect to a pressure engine having an internal chamber with an internal pressure that is a sub-atmospheric pressure, the internal pressure of the internal chamber of the pressure engine is sub-atmospheric relative to the external atmospheric pressure in the environment in which the pressure engine is used, as described herein. The term “sub-atmospheric pressure environment” is used herein to mean an environment such as a geographic location or contained physical structure that is below external atmospheric pressure in the environment.

The term “fluid” is used herein to refer to anything having fluidic properties, including gases (e.g., air) and/or liquids (e.g., exudate, blood, puss).

The term “automatically” is used herein to mean without human intervention.

The word “exemplary” is used herein to mean serving as a non-limiting example, instance, or illustration. Any embodiment described herein as “exemplary” or as an “example” is not necessarily to be construed as preferred or advantageous over other embodiments. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein can be applied to other aspects. Thus, the claims are not intended to be limited to the aspects shown herein but are to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neutral gender (e.g., her and its) and vice versa. Headings and subheadings, if any, are used for convenience only and do not limit the subject disclosure.

What is claimed is:

1. A negative pressure wound therapy system, comprising:
a dressing configured to be positioned on a wound site, the dressing comprising:
a gasket configured to form an airtight seal surrounding the wound site; and
a first adhesive configured to attach the dressing to skin surrounding the wound site;
a hub configured to have aspirational attachment to the dressing; and
a sealed pressure engine pre-evacuated to a sub-atmospheric pressure and configured to have aspirational attachment to the hub such that the pressure engine is configured to aspirate fluid away from the dressing via a fluid channel extending from the dressing, through the hub, to the pressure engine.

2. The negative pressure wound therapy system of claim 1, wherein the dressing comprises a drape comprising a first side configured to face toward the wound site and a second side configured to face away from the wound site wherein at least a portion of the first side comprises a metallic film.

3. The negative pressure wound therapy system of claim 2, wherein the drape comprises a portion extending beyond the gasket and comprising the first adhesive.

4. The negative pressure wound therapy system of claim 1, wherein at least a portion of (a) an interior surface of the pressure engine or (b) an exterior surface of the pressure engine comprises a metallic film.

5. The negative pressure wound therapy system of claim 1, wherein the gasket comprises a central opening and the dressing further comprises a wound contact structure disposed within the central opening of the gasket.

6. The negative pressure wound therapy system of claim 1, further comprising a tubing extending between and removably attached to both the dressing and the hub, the tubing thereby aspirationally attaching the hub to the dressing.

7. The negative pressure wound therapy system of claim 1, wherein:
the dressing comprises a plurality of dressings,
the tubing comprises a plurality of tubings, each tubing of the plurality of tubings corresponding to a respective one of the plurality of dressings, and
the hub comprises a plurality of tubing interfaces, each tubing interface of the plurality of tubing interfaces being configured to be removably attached to a respective one of the plurality of tubings, each tubing of the plurality of tubings thereby aspirationally attaching a respective one of the plurality of dressings to the hub.

8. The negative pressure wound therapy system of claim 1, wherein the hub comprises a second adhesive configured to attach the hub to the skin at a different location than the wound site.

9. The negative pressure wound therapy system of claim 1, wherein the hub is directly attached to the dressing.

10. The negative pressure wound therapy system of claim 1, wherein the pressure engine is configured to be physically attached to the hub and at least one of the hub and the pressure engine is configured to be selectively moved relative to the other of the hub and the pressure engine between (a) a first position in which the pressure engine is physically attached to the hub but not aspirationally attached to the hub and (b) a second position in which the pressure engine is both physically attached to the hub and aspirationally attached to the hub.

11. The negative pressure wound therapy system of claim 1, wherein:
the hub comprises a pressure engine interface, and
the pressure engine is configured to automatically aspirate fluid away from the dressing in response to being aspirationally attached to the pressure engine interface of the hub.

12. The negative pressure wound therapy system of claim 11, wherein the pressure engine comprises a hub interface, which together with the pressure engine interface is configured to form the fluid channel.

13. The negative pressure wound therapy system of claim 12, wherein either the pressure engine interface of the hub or the hub interface of the pressure engine comprises a seal and the other of the pressure engine interface of the hub and the hub interface of the pressure engine comprises an engagement feature configured to puncture the seal such that an airtight seal is formed between the pressure engine and the hub and the pressure engine is configured to automatically aspirate fluid away from the dressing in response to puncturing the seal.

14. The negative pressure wound therapy system of claim 12, wherein one of the pressure engine interface of the hub and the hub interface of the pressure engine comprises a valve or a membrane and the other of the pressure engine interface of the hub and the hub interface of the pressure engine comprises an engagement feature configured to open the valve or puncture the membrane, such that an airtight seal is formed between the pressure engine and the hub and the pressure engine is configured to automatically aspirate fluid away from the dressing in response to opening the valve or puncturing the membrane.

15. The negative pressure wound therapy system of claim 1, wherein:
the hub comprises one or more guides disposed atop the hub for aligning the pressure engine and the hub; and/or
the pressure engine comprises one or more guides disposed beneath the pressure engine for aligning the pressure engine and the hub.

16. A method for applying the negative pressure wound therapy system of claim 1, the method comprising:
positioning the dressing on the wound site such that the gasket forms an airtight seal surrounding the wound site and the first adhesive attaches the dressing to the skin surrounding the wound site;
aspirationally attaching the hub to the dressing;
aspirationally attaching the pressure engine to the hub; and
aspirating, using the pressure engine, fluid away from the dressing via the fluid channel extending from the dressing, through the hub, to the pressure engine.

17. The method of claim 16, wherein the pressure engine is a first pressure engine, the method further comprising:
aspirationally detaching the first pressure engine from the hub after aspirating fluid away from the dressing using the first pressure engine;
aspirationally attaching a second pressure engine pre-evacuated to a sub-atmospheric pressure to the hub; and
aspirating, using the second pressure engine, fluid away from the dressing.

18. The method of claim 16, further comprising:
selectively moving at least one of the hub and the pressure engine relative to the other of the hub and the pressure engine between (a) a first position in which the pressure engine is physically attached to the hub but not aspirationally attached to the hub and (b) a second position in which the pressure engine is both physically attached to the hub and aspirationally attached to the hub.

19. A method for manufacturing a negative pressure wound therapy system, the method comprising:
sealing a pressure engine having an internal chamber in a first environment such that the internal chamber of the pressure engine is at an internal pressure equivalent to the external atmospheric pressure in the first environment; and
moving the pressure engine from the first environment to a second environment different from the first environment, such that the internal pressure of the internal chamber of the pressure engine is sub-atmospheric relative to the external atmospheric pressure in the second environment.

20. A method for manufacturing a negative pressure wound therapy system, the method comprising:
creating or providing a controlled pressure environment; and
sealing a pressure engine having an internal chamber in the controlled pressure environment such that the internal chamber of the pressure engine is at a sub-atmospheric pressure equivalent to an air pressure of the controlled pressure environment.
